(19) United States
(12) Patent Application Publication
(10) Pub. No.: US 2018/0177725 A1
(43) Pub. Date: Jun. 28, 2018

(54) CELL MEMBRANE-FORMED NANOSCALE VESICLES AND METHODS OF USING THEREOF

(71) Applicant: WASHINGTON STATE UNIVERSITY, Pullman, WA (US)

(72) Inventor: Zhenjia WANG, Spokane, WA (US)

(21) Appl. No.: 15/759,278

(22) PCT Filed: Sep. 12, 2016
(86) PCT No.: PCT/US16/51288

§ 371 Date: Mar. 12, 2018

Related U.S. Application Data
(60) Provisional application No. 62/216,474, filed on Sep. 10, 2015.

(51) Int. Cl. A61K 9/127; A61K 35/28; A61K 35/74; A61K 38/17; A61K 45/00

(52) U.S. Cl. CPC A61K 9/127; A61K 35/28; A61K 45/00; A61K 38/1774; A61K 35/74

(57) ABSTRACT

The present disclosure provides vesicle composition comprising a cell membrane and an agent for the treatment and identification of diseases. Methods of treatment and methods of making a vesicle composition are also provided.

### CROSS-REFERENCE TO RELATED APPLICATION

This application claims the benefit under 35 USC § 119(e) of U.S. Provisional Application Ser. No. 62/216,474, filed on Sep. 10, 2015, the entire disclosure of which is incorporated herein by reference.

### GOVERNMENT RIGHTS

This invention was made with government support under Grant Nos. K25HL111157 and 1RO1GM116823, awarded by National Institutes of Health. The government has certain rights in the invention.

### TECHNICAL FIELD

The present disclosure provides vesicle composition comprising a cell membrane and an agent for the treatment and identification of diseases. Methods of treatment and methods of making a vesicle composition are also provided.

### BACKGROUND AND SUMMARY OF THE INVENTION

Nanomedicine to specifically treat diseases is premised on the ability to achieve targeted drug delivery and desirable drug retention in a specific location. However, current approaches are insufficient for actively delivering nanoparticles directly to a desired location. In this regard, creation of a nanoparticle that is both capable of delivering agents and specific to the cell type to be affected is highly desirable. However, creation of membrane-enclosed vesicles (such as extracellular vesicles) can result in the inclusion of undesirable components on or within the membrane-enclosed vesicles, thus rendering their use of safe drug delivery questionable.

Accordingly, the present disclosure provides a novel platform for exploiting a diseased cell as a building block to create cell membrane-formed nanosized vesicles. The novel platform utilizes a general method to create cell membrane-formed vesicles using a mechanical force generated by nitrogen cavitation, which disrupts cells and maintains intact biological functions of membrane molecules.

For example, the vascular endothelium lining the lumen of blood vessels regulates a variety of functions, including expression of proteins and release of plasma growth factors, and regulation of tissue fluid homeostasis via junctional molecules. A monolayer of endothelium selectively transports plasma molecules and nanoparticles across vessel walls through transcytosis. Moreover, the vascular endothelium plays a central role in immunity to respond infection and tissue damage. Because endothelium essentially governs the systemic physiology, dysfunctional endothelium is underlying components of most diseases, for example cancer, atherosclerosis, sepsis, autoimmune disease, and acute lung inflammation/injury, thus targeting of diseased vasculature might be an effective means to improve the current therapies.

Vascular inflammation is a feature of immune response that is a movement of immune cells from one location to another, and the mechanism underlying the migration is regulated by intercellular adhesion molecules. At inflammation sites endothelium rapidly upregulates intercellular adhesion molecule 1 (ICAM-1) mainly through the NF-κB pathway to recruit leukocytes. Neutrophils, a type of polymorphonuclear leukocytes and the most abundant circulating leukocytes in human, are a central player in acute inflammation induced by infection or tissue damage. They are the first to migrate to the inflammatory location and are capable of eliminating pathogens, but dysregulated neutrophil recruitment and excessive vascular inflammation could cause organ failure and damage, such as acute lung inflammation and injury.

To target inflamed vasculature, nanoparticles are commonly engineered by conjugating anti-ICAM-1 or peptides to the surface of nanoparticles. However, the conjugation can prevent their specificity and affinity to the target, in particular when the nanoparticles are administrated in vivo. When inflammation occurs, neutrophils highly express integrin β2, which binds to endothelial cells via ICAM-1 molecules. Accordingly, the present disclosure provides cells such as activated neutrophils as a building block to generate nanosized vesicles derived from neutrophil membrane using a mechanical force enabling to rapidly break a neutrophil apart. The resulting vesicles highly bind to activated endothelium in vitro and in vivo because the vesicles possess intact integrin β2 that interacts with ICAM-1 expressed on activated endothelium. This disclosure establish a novel approach in which a given disease fuels the design of nanotherapeutics produced by their own diseased tissue, and can be adaptable to various disease states in which new therapies and diagnostics are desired.

The present disclosure provides vesicle composition comprising a cell membrane and an agent for the targeted treatment and identification of diseases. Advantageously, vesicle compositions are comprised of cell membranes of the cell type or cell types for which they are targeted, thus providing a specific therapy to a patient in need. According to the methods of the present disclosure, a range of therapeutics can be targeted with the vesicle compositions, thus improving therapies of various diseases for which the vesicle compositions can be made. For example, diseases to be treated include but are not limited to inflammation, cancer, metabolic disorders, cardiovascular, and infectious diseases.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIGS. 1A-1D show generation of cell-membrane-formed vesicles and their characteristics. (FIG. 1A) Schematic shows a process to generate a uniform size of vesicles including cell disruption, differential centrifugation and extrusion. (FIG. 1B) Quantification of DNA and proteins contained in each step of centrifugation (experiments n=3). (FIG. 1C) Average size of HL 60 cell-membrane formed vesicles (HV) and erythrocyte vesicles (EV) using dynamic light scattering and TEM (insect). Scale bar, 200 nm. (FIG. 1D) The Zeta potential of HL 60 and erythrocyte cells and their vesicles.

FIGS. 2A-2D show the role of Integrin β₂ in the internalization of HV vesicles in Human umbilical vein endothelial cells (HUVECs). (FIG. 2A) Western blot of HL 60 and erythrocyte cells and their vesicles. (FIG. 2B) The integrin β₂ expression against actin in HL 60 cells and their vesicles quantified from Western blot. (n=3). (FIG. 2C) Fluorescence confocal images of inside of cells which were incubated with Dil-fluorescently-labeled vesicles (HV or EV). HUVECs were treated with 100 ng/ml of TNF-α. (FIG. 2D) Uptake of HV or EV nanovesicles by HUVECs obtained from flow cytometry. At 3 hours after HUVECs treatment with TNF-α (100 ng/ml), they were incubated with Dil-fluorescently-labeled vesicles, and then flow cytometry was used to measure the mean fluorescence intensity per cell. The binding of nanovesicles to HUVECs was performed at 0° C. and followed with washing an increase in temperature to 37° C. To inhibit the binding of integrin β₂ to ICAM-1, HUVECs were pre-treated with anti-ICAM-1 antibody (10 μg/ml). ** represents p value<0.01. All data expressed as Mean±SD.

FIGS. 3A-3E show the role of activation of endothelium vessel for the adherence of HV vesicles in vivo. (FIG. 3A) Intravital image of a cremaster venule of a live mouse intrascrotally treated with TNF-α (0.5 (μg) after infusion of DiO-fluorescently-labeled HV vesicles (green) and Alex-Fluor-647-labeled anti-CD31(pink). The image was taken by excitation laser at 488 nm and 640 nm using AR1⁺ resonant-scanning confocal microscope. 0.1 mg of HV vesicles and 2.5 μg of CD31 antibody were intravenously administered. (FIG. 3B) Intravital images of a cremaster venule activated with TNF-α after infusion of DiL-fluorescently-labeled HV and DiO-fluorescently-labeled EV vesicles at 0.1 mg/mouse. (FIG. 3C) Intravital images of a cremaster venule without TNF-α treatment at the same condition as FIG. 3B. Approximately 1 hour after tail vein injection of the vesicles, the cremaster venules were surgerically exposed for intravital microscopy. This procedure kept the venule vessels resting when the vesicles were administered. (FIG. 3D) Quantification of adherence of the vesicles to venule vessels based on intravital images using Nikon software (NIS Elements). The vessel size was from 20-30 μm in diameter. 3 mice were used per group. (FIG. 3E) Intravital image shows HV vesicles accumulated in the inflamed area where neutrophils existed. Alex-fluor-488-anti-Gr antibody and Dil-fluorescently-labeled HV vesicles were simultaneously injected to a live mouse intrascrotally treated with TNF-α.

FIGS. 4A-4E show TPCA-1-loaded HV vesicles dramatically attenuate vascular inflammation in vitro and vivo. (FIG. 4A) TPCA-1-loaded HV vesicles (HV-TPCA-1) decreased ICAM-1 expression of HUVECs after treated with TNF-α compared with TPCA-1 loaded EV vesicles (EV-TPCA-1). Western blot shown in the insect. (FIGS. 4B-4E) The diagram shows the experimental protocol above the figures. Numbers of neutrophils (FIG. 4B), and concentrations of TNF-α (FIG. 4C), IL-6 (FIG. 4D), and proteins (FIG. 4E) present in BALF 13 hours after intravenous injection of HBSS, TPCA-1 solution, EV-TPCA-1 and HV-TPCA-1 vesicles in mice 4 hours after LPS challenge (10 mg/kg). The dose of TPCA-1 was 0.33 mg/kg and 1 mg/kg, respectively. For TNF-α and IL-6, the dose of TPCA-1 was 1 mg/kg to a mouse. *, **, and *** represent p value of <0.05, 0.01 and 0.001 in two-way t-Test.

FIG. 5 shows that the sonication approach can change the pH values inside HL 60 vesicles. Incubating a pH tracker L7526 with HBSS, and PB (sodium phosphate at pH=3.5), and vesicle 1 and vesicle 2 sonicated at the low power and high power for 2 minutes, respectively

FIG. 6 shows caffeine loading in HL 60 vesicles with a pH=3.5 inside vesicles. Sample 1: caffeine (10 mg/ml) in HBSS buffer (pH=7.5); Sample 2: caffeine (10 mg/ml) in HBSS buffer (pH=7.5), and sonication of solution; Sample 3: caffeine (10 mg/ml) in the buffer (pH=9). Incubation with the vesicles for 2 hours.

FIGS. 7A-7B show a pH sensor of SNARF-1 AM was trapped in HL 60 cells and their vesicles. FIG. 7A: Before and after SNARF-1 AM was incubated with HL 60 cells after centrifugation. FIG. 7B: Before and after SNARF-1 AM was incubated with HL 60 cells after the cells were disrupted by nitrogen cavitation.

FIG. 8 shows pH value changes after the drug loading. The pH values were measured using SNARF-1 AM in HL 60 cells (internal pH of cells), and inside of HL 60 vesicles (internal pH of vesicles) and after loading of piceatannol (1 mg/ml in HBSS with a pH=4). The emission ratio was measured at 640 nm/580 nm to obtain real pH values.

FIG. 9 shows loading of bromophenol blue. After HL 60 vesicles were made at pH=9.5, bromophenol blue was loaded at the different pH values. By changing the pH=4, the loading efficiency can dramatically be increased.

FIGS. 10A-10D show a broad size range of disrupted cell lysates after each step of centrifugation using HL 60 cells. (FIG. 10A) The whole cell lysate after cell disruption using nitrogen cavitation at 350 psi. (FIG. 10B) Supernatant after centrifugation at 2,000 g. (FIG. 10C) A pellet after centrifugation at 100,000 g. (FIG. 10D) The pellet extruded through a membrane with pores of 200 nm in diameter.

FIG. 11 shows the size of HV and EV vesicles measured using dynamic light scattering.

FIG. 12 shows Integrin β2 was upregulated in HL 60 cells after treatment with DMSO (1.3% v/v).

FIG. 13 shows ICAM-1 was upregulated in HUVECs after treatment with TNF-α (100 ng/ml) for 3 hours.

FIGS. 14A-14B show detection of TPCA-1 loaded in the vesicles using HPLC. The TPCA-1 signal was detected by UV-HPLC at 310 nm (FIG. 14A) and Mass Spectrometer confirming that the mass of TPCA-1 (FIG. 14B). The result indicates the HPLC measured TPCA-1 molecules.

FIG. 15 shows the Western blot of HL 60 nanovesicles produced by nitrogen cavitation and EVs (extracellular vesicles) made from HL 60 culture medium.

FIGS. 16A-16B show the quantification of biomarkers from HL 60 nanovesicles produced by nitrogen cavitation (FIG. 16A) and EVs made from HL 60 culture medium (FIG. 16B).

FIGS. 17A-17B show the DNA amount in cell-formed nanovesicles (FIG. 17A) and production efficiency of cell-formed nanovesicles (FIG. 17B). HL 60 nanovesicles were made using nitrogen cavitation (HVs) and from HL 60 cell culture medium (EVs).

FIGS. 18A-18B show the size (FIG. 18A) and the surface charge (FIG. 18B) of cell-membrane-formed nanovesicles using dynamic light scattering. HL 60 nanovesicles made using nitrogen cavitation (HVs) and from HL 60 cell culture medium (EVs). Piceatannol, an anti-inflammation drug, was loaded in HVs (Piceatannol Vesicles).

FIGS. 19A-19C show pH-driven loading of piceatannol inside a nanovesicle. (FIG. 19A) A concept of loading piceatannol in HL 60 nanovesicles based on the mechanism of pH gradient. (FIG. 19B) pH values of HL 60 cells and their nanovesicles made in different pH buffers. HL 60 cells were incubated with SNARF-1-AM (pH indicator), and then the cells were disrupted by nitrogen cavitation to form nanovesicles in the buffer at pH=7.4 or pH=9.0. (FIG. 19C) The piceatannol loading efficiency is driven by a pH gradient. The buffer outside nanovesicles was kept at pH=7.4, and the inside of nanovesicles was pH=7.4 or pH=9.0.

FIG. 20 shows the increase in mouse survival in a sepsis model. Approximately 2 hours after LPS injection (22 mg/kg), HBSS, piceatannol (3 mg/kg) and piceatannol-loaded nanovesicles (piceatannol of 3 mg/kg) were intravenously administrated to the mice. The p value represents the difference between nanovesicle group and piceatannol treatment group or sham group (HBSS).

FIGS. 21A-21B show the size (FIG. 21A) and Zeta potential (FIG. 21B) of both human neutrophil and mouse neutrophil nanovesicles made using nitrogen cavitation.

FIG. 22 shows the polydispersity of HV and EV nanovesicles as characterized by dynamic light scattering. The polydispersity index (PDI) shows that the nanovesicles appear moderately uniform, consistent with Cyro-TEM images. (n=3).

FIG. 23 shows the change in size of HV nanovesicles over time. The HV nanovesicles were stored at 4° C. and at defined time points, the vesicle size was measured using dynamic light scattering. The size slightly increased with time 6 days after the storage at 4° C. (n=3).

FIG. 24 shows that lyophilization of HV nanovesicles retains nanovesicle size. The 4 wt % of sorbitol was added in nanovesicle suspension during the lyophilization. The nanovesicle sizes were measured after the reconstruction of lyophilized nanovesicles and their sonication. Vesicle size did not change after lyophilzation and sonication. (n=3)

FIG. 25 shows the stability of TPCA-1-loaded nanovesicles over time. After TPCA-1 was loaded in HV nanovesicles, the nanovesicles were stored at 4° C. At defined time points, the nanovesicle suspension was centrifuged at 100,000 g for 30 minutes, and TPCA-1 in the supernatant was quantified using HPLC. The TPCA-1 was retained in nanovesicles for a week after the drug was slightly released in the initial 2 days. (n=3).

FIG. 26 shows a Western blot (10% SDS-PAGE) of HL 60 cell lysis and their products after each centrifugation staining with commassie blue G-250. Line 1: whole cell lysate; Line 2: Supernatant after the centrifugation at 100,000 g; Line 3: HV nanovesicles.

FIG. 27 shows a Western blot (10% SDS-PAGE) of HL 60 cell lysis and their products after each centrifugation. Line 1: Molecular Marker; Line 2: Whole cell lysate; Line 3: Supernatant after the centrifugation at 100,000 g; Line 4: HV vesicles.

FIGS. 28A-28B show bio-distribution of HV nanovesicles in the mice with or without (w/o) LPS challenge. FIG. 28A shows 1 hour after and FIG. 28B shows 10 hours after i.v. injection of HV nanovesicles fluorescently labeled with DiD in the mice, 3 hours after lung LPS instillation. * and ** represent p value<0.05 and <0.01. The tissues were collected and homogenized for fluorescence measurement.

FIG. 29 shows the size of Pseudomonas Aeruginosa and vesicle compositions made from Pseudomonas Aeruginosa membranes. Dynamic light scattering was used to measure the size.

FIGS. 30A-30B show cryo-TEM images of Pseudomonas Aeruginosa (FIG. 30A); vesicle compositions made using nitrogen cavitation (FIG. 30B); outer membrane vesicles (OMVs) from culture medium (FIG. 30C); and the quantitative measurement of membrane thickness for Pseudomonas Aeruginosa, vesicle compositions, and OMVs (FIG. 30D).

The following numbered embodiments are contemplated and are non-limiting:
1. A vesicle composition comprising a cell membrane and an agent.
2. The vesicle composition of clause 1, wherein the vesicle composition further comprises a targeting molecule.
3. The vesicle composition of clause 2, wherein the targeting molecule is an intact targeting molecule.
4. The vesicle composition of clause 2 or clause 3, wherein the targeting molecule is a membrane molecule.
5. The vesicle composition of any one of clauses 1 to 4, wherein the targeting molecule is on the surface of the vesicle composition.
6. The vesicle composition of any one of clauses 1 to 5, wherein the targeting molecule is derived from the cell membrane.
7. The vesicle composition of any one of clauses 1 to 6, wherein the targeting molecule is a cell adhesion molecule.
8. The vesicle composition of clause 7, wherein the cell adhesion molecule is an intercellular adhesion molecule.
9. The vesicle composition of clause 7, wherein the cell adhesion molecule is integrin β2.
10. The vesicle composition of clause 7, wherein the cell adhesion molecule is ICAM-1.
11. The vesicle composition of any one of clauses 1 to 10, wherein the vesicle composition is a nanovesicle.
12. The vesicle composition of any one of clauses 1 to 11, wherein the vesicle composition has an average diameter from about 40 nm to about 500 nm.
13. The vesicle composition of any one of clauses 1 to 11, wherein the vesicle composition has an average diameter from about 100 nm to about 300 nm.
14. The vesicle composition of any one of clauses 1 to 11, wherein the vesicle composition has an average diameter from about 80 nm to about 200 nm.
15. The vesicle composition of any one of clauses 1 to 11, wherein the vesicle composition has an average diameter of about 100 nm.
16. The vesicle composition of any one of clauses 1 to 11, wherein the vesicle composition has an average diameter of about 200 nm.
17. The vesicle composition of any one of clauses 1 to 11, wherein the vesicle composition has an average diameter of about 300 nm.
18. The vesicle composition of any one of clauses 1 to 11, wherein the vesicle composition has an average diameter of about 400 nm.
19. The vesicle composition of any one of clauses 1 to 11, wherein the vesicle composition has an average diameter of about 500 nm.
20. The vesicle composition of any one of clauses 1 to 19, wherein the vesicle composition is substantially free of one or more intracellular organelles.
21. The vesicle composition of clause 20, wherein the intracellular organelles comprise one or more of endoplasmic reticulum, mitochondria, lysosomes, and Golgi bodies.
22. The vesicle composition of any one of clauses 1 to 21, wherein the cell membrane is a neutrophil cell membrane.
23. The vesicle composition of clause 22, wherein the neutrophil cell membrane is a HL60 cell membrane.
24. The vesicle composition of clause 22, wherein the neutrophil cell membrane is a human neutrophil cell membrane.
25. The vesicle composition of clause 22, wherein the neutrophil cell membrane is a rodent neutrophil cell membrane.
26. The vesicle composition of any one of clauses 1 to 21, wherein the cell membrane is a cancer cell membrane.
27. The vesicle composition of clause 26, wherein the cancer cell membrane is a HL60 cell membrane.
28. The vesicle composition of clause 26, wherein the cancer cell membrane is a HeLa cell membrane.
29. The vesicle composition of clause 26, wherein the cancer cell membrane is a 3LL cell membrane.
30. The vesicle composition of clause 26, wherein the cancer cell membrane is selected from the group consisting of a NCI-H1299 cell membrane, a HCC70 cell membrane, a RWPE-1 cell membrane, a CWR-R1 cell membrane, a C4-2 cell membrane, a HEK293 cell membrane, a PC-3 cell membrane, a SKOV3 cell membrane, a MDA PCa 2b cell membrane, a LNCaP95 cell membrane, a MCF-7 cell membrane, a SGBS cell membrane, a C4-2b cell membrane, a NHLF cell membrane, a LNCaP-C81 cell membrane, a Hela cell membrane, a VCaP cell membrane, a 293T cell membrane, a MDA-MB-468 cell membrane, a ATCC-231 (MDA-MB-231ATCC® HTB-26™) cell membrane, a ATCC-LNCaP (LNCaP clone FGCATCC® CRL-1740™H) cell membrane, a RVE cell membrane, a LNCaP (LNCaP clone FGCATCC® CRL-1740™) cell membrane, a NDA157 (MDA-MB-157) cell membrane, and a PNT1A cell membrane.
31. The vesicle composition of any one of clauses 1 to 21, wherein the cell membrane is an endothelial cell membrane.
32. The vesicle composition of clause 31, wherein the endothelial cell membrane is a HUVEC cell membrane.
33. The vesicle composition of clause 31, wherein the endothelial cell membrane is a human lung microvascular endothelial cell.
34. The vesicle composition of any one of clauses 1 to 21, wherein the cell membrane is an epithelial cell membrane.
35. The vesicle composition of any one of clauses 1 to 21, wherein the cell membrane is a bacterial cell membrane.
36. The vesicle composition of clause 35, wherein the bacterial cell membrane is a gram-negative bacterial cell membrane.
37. The vesicle composition of clause 36, wherein the gram-negative bacterial cell membrane is selected from the group consisting of E. coli, Neisseria gonorrhoeae, Chlamydia trachomatis, Yersinia pestis, Proteus, and Leptospiria.
38. The vesicle composition of clause 35, wherein the bacterial cell membrane is a gram-positive bacterial cell membrane.
39. The vesicle composition of clause 38, wherein the gram-positive bacterial cell membrane is selected from the group consisting of Mycoplasma, Bacillus, Staphylococcus, Streptomyces, and Enterococcus.
40. The vesicle composition of clause 35, wherein the bacterial cell membrane is an Escherichia coli cell membrane.
41. The vesicle composition of clause 35, wherein the bacterial cell membrane is a Pseudomonas cell membrane.
42. The vesicle composition of any one of clauses 1 to 21, wherein the cell membrane is a viral cell membrane.
43. The vesicle composition of any one of clauses 1 to 21, wherein the cell membrane is a primary cell membrane.
44. The vesicle composition of any one of clauses 1 to 21, wherein the cell membrane is an immune cell membrane.
45. The vesicle composition of any one of clauses 1 to 21, wherein the cell membrane is a human cell membrane.
46. The vesicle composition of any one of clauses 1 to 21, wherein the cell membrane is a rodent cell membrane.
47. The vesicle composition of any one of clauses 1 to 46, wherein the cell membrane forms the majority of the vesicle composition.
48. The vesicle composition of any one of clauses 1 to 46, wherein the cell membrane is more than 50% of the vesicle composition by weight.
49. The vesicle composition of any one of clauses 1 to 46, wherein the cell membrane is about 50% of the vesicle composition by weight.
50. The vesicle composition of any one of clauses 1 to 46, wherein the cell membrane is about 75% of the vesicle composition by weight.
51. The vesicle composition of any one of clauses 1 to 46, wherein the cell membrane is between 50%-75% of the vesicle composition by weight.
52. The vesicle composition of any one of clauses 1 to 51, wherein the vesicle composition is formed via nitrogen cavitation.
53. The vesicle composition of any one of clauses 1 to 52, wherein the vesicle composition is a cell-targeted vesicle composition.
54. The vesicle composition of clause 53, wherein the cell-targeting corresponds to the cell type of the cell membrane.
55. The vesicle composition of any one of clauses 1 to 54, wherein the agent is a therapeutic agent.
56. The vesicle composition of clause 55, wherein the therapeutic agent is acidic.
57. The vesicle composition of clause 55, wherein the therapeutic agent is basic.
58. The vesicle composition of any one of clauses 55 to 57, wherein the therapeutic agent is an antibiotic.
59. The vesicle composition of any one of clauses 55 to 57, wherein the therapeutic agent is an anti-inflammatory agent.
60. The vesicle composition of clause 59, wherein the anti-inflammation agent is selected from the group consisting of anti-inflammatory glucocorticoids, NF-kB inhibitors, p38MAP kinase inhibitors, Syk/Zap kinase inhibitors, and siRNA oligonucleotides against genes involved in pro-inflammation.
61. The vesicle composition of clause 59, wherein the anti-inflammation agent is selected from the group consisting of TPCA-1 (2-[(Aminocarbonyl)amino]-5-(4-fluorophenyl)-3-thiophenecarboxamide), PS-1145 (N-(6-Chloro-9H-pyrido[3,4-b]indol-8-yl)-3-pyridinecarboxamide dihydrochloride), ML-120B (N-(6-Chloro-7-methoxy-9H-pyrido[3,4-b]indol-8-yl)-2-methyl-3-pyridinecarboxamide), SC-514 (4-Amino-[2′,3′-bithiophene]-5-carboxamide), IMD-0354 (N-[3,5-Bis(trifluoromethyl)phenyl]-5-chloro-2-hydroxybenzamide), BMS-345541 (N-(1,8-Dimethylimidazo[1,2-a]quinoxalin-4-yl)-1,2-ethanediamine hydrochloride), and Bay 11-7085 ((E)-3-(4-t-Butylphenylsulfonyl)-2-propenenitrile).
62. The vesicle composition of clause 59, wherein the anti-inflammation agent is an anti-inflammatory glucocorticoid.
63. The vesicle composition of clause 59, wherein the anti-inflammation agent is an NF-kB inhibitor.
64. The vesicle composition of clause 59, wherein the anti-inflammation agent is a p38MAP kinase inhibitor.
65. The vesicle composition of clause 59, wherein the anti-inflammation agent is a Syk/Zap kinase inhibitor.
66. The vesicle composition of clause 59, wherein the anti-inflammation agent is an siRNA oligonucleotide against genes involved in pro-inflammation.
67. The vesicle composition of clause 59, wherein the anti-inflammatory agent is piceatannol.
68. The vesicle composition of any one of clauses 55 to 57, wherein the therapeutic agent is an anti-cancer agent.
69. The vesicle composition of any one of clauses 55 to 57, wherein the therapeutic agent is an NF-κB inhibitor.
70. The vesicle composition of clause 69, wherein the NF-κB inhibitor is TPCA-1.
71. The vesicle composition of any one of clauses 1 to 54, wherein the agent is a diagnostic agent.
72. The vesicle composition of clause 71, wherein the diagnostic agent is acidic.
73. The vesicle composition of clause 71, wherein the diagnostic agent is basic.
74. The vesicle composition of any one of clauses 71 to 73, wherein the diagnostic agent is selected from the group consisting of fluorescent probes or tags, isotope probes or tags, antibody probes or tags, antigen probes or tags, enzyme probes or tags, dye probes or tags, biotin-binding protein probes or tags, and bioluminescence reporter probes or tags.
75. The vesicle composition of any one of clauses 71 to 73, wherein the diagnostic agent is a fluorescent probe or tag.
76. The vesicle composition of any one of clauses 71 to 73, wherein the diagnostic agent is an isotope probe or tag.
77. The vesicle composition of any one of clauses 71 to 73, wherein the diagnostic agent is an antibody probe or tag.
78. The vesicle composition of any one of clauses 71 to 73, wherein the diagnostic agent is an antigen probe or tag.
79. The vesicle composition of any one of clauses 71 to 73, wherein the diagnostic agent is an enzyme probe or tag.
80. The vesicle composition of any one of clauses 71 to 73, wherein the diagnostic agent is a dye probe or tag.
81. The vesicle composition of any one of clauses 71 to 73, wherein the diagnostic agent is a biotin-binding protein probe or tag.
82. The vesicle composition of any one of clauses 71 to 73, wherein the diagnostic agent is a bioluminescence reporter probe or tag.
83. The vesicle composition of any one of clauses 71 to 73, wherein the diagnostic agent is a photosensitizer.
84. The vesicle composition of clause 83, wherein the photosensitizer is a derivative of porphyrin and/or chlorophyli.
85. The vesicle composition of clause 83, wherein the photosensitizer is selected from the group consisting of Allumera, Photofrin, Visudyne, Levulan, Foscan, Metvix, Hexvix, Cysview, and Laserphyrin.
86. A method of treating a disease in a patient in need thereof, said method comprising the step of administering a vesicle composition comprising a cell membrane and an agent to the patient, wherein the administration of the vesicle composition reduces one or more symptoms associated with the disease.
87. The method of clause 86, wherein the disease is an inflammatory disease.
88. The method of clause 87, wherein the inflammatory disease is an acute inflammatory disease.
89. The method of clause 87, wherein the inflammatory disease is a chronic inflammatory disease.
90. The method of clause 87, wherein the inflammatory disease is cancer.
91. The method of clause 87, wherein the inflammatory disease is sepsis.
92. The method of clause 87, wherein the inflammatory disease is a lung injury.
93. The method of clause 92, wherein the lung injury is an acute lung injury.
94. The method of clause 92, wherein the lung injury is a chronic lung injury.
95. The method of clause 86, wherein the disease is an infection.
96. The method of clause 95, wherein the infection is a bacterial infection.
97. The method of clause 95, wherein the infection is a viral infection.
98. The method of clause 95, wherein the infection is a fungal infection.
99. The method of any one of clauses 86 to 98, wherein the administration is a parenteral administration.
100. The method of clause 99, wherein the parenteral administration is an intravenous administration.
101. The method of any one of clauses 86 to 100, wherein the administration reduces ICAM-1 expression in the patient.
102. The method of any one of clauses 86 to 101, wherein the vesicle composition further comprises a targeting molecule.
103. The method of clause 102, wherein the targeting molecule is an intact targeting molecule.
104. The method of clause 102 or clause 103, wherein the targeting molecule is a membrane molecule.
105. The method of any one of clauses 102 to 104, wherein the targeting molecule is on the surface of the vesicle composition.
106. The method of any one of clauses 102 to 105, wherein the targeting molecule is derived from the cell membrane.
107. The method of any one of clauses 102 to 106, wherein the targeting molecule is a cell adhesion molecule.
108. The method of clause 107, wherein the cell adhesion molecule is an intercellular adhesion molecule.
109. The method of clause 107, wherein the cell adhesion molecule is integrin β2.
110. The method of clause 107, wherein the cell adhesion molecule is ICAM-1.
111. The method of any one of clauses 86 to 110, wherein the vesicle composition is a nanovesicle.
112. The method of any one of clauses 86 to 111, wherein the vesicle composition has an average diameter from about 40 nm to about 500 nm.
113. The method of any one of clauses 86 to 111, wherein the vesicle composition has an average diameter from about 100 nm to about 300 nm.
114. The method of any one of clauses 86 to 111, wherein the vesicle composition has an average diameter from about 80 nm to about 200 nm.
115. The method of any one of clauses 86 to 111, wherein the vesicle composition has an average diameter of about 100 nm.
116. The method of any one of clauses 86 to 111, wherein the vesicle composition has an average diameter of about 200 nm.
117. The method of any one of clauses 86 to 111, wherein the vesicle composition has an average diameter of about 300 nm.
118. The method of any one of clauses 86 to 111, wherein the vesicle composition has an average diameter of about 400 nm.
119. The method of any one of clauses 86 to 111, wherein the vesicle composition has an average diameter of about 500 nm.
120. The method of any one of clauses 86 to 119, wherein the vesicle composition is substantially free of one or more intracellular organelles.
121. The method of clause 120, wherein the intracellular organelles comprise one or more of endoplasmic reticulum, mitochondria, lysosomes, and Golgi bodies.
122. The method of any one of clauses 86 to 121, wherein the cell membrane is a neutrophil cell membrane.
123. The method of clause 122, wherein the neutrophil cell membrane is a HL60 cell membrane.
124. The method of clause 122, wherein the neutrophil cell membrane is a human neutrophil cell membrane.
125. The method of clause 122, wherein the neutrophil cell membrane is a rodent neutrophil cell membrane.
126. The method of any one of clauses 86 to 121, wherein the cell membrane is a cancer cell membrane.
127. The method of clause 126, wherein the cancer cell membrane is a HL60 cell membrane.
128. The method of clause 126, wherein the cancer cell membrane is a HeLa cell membrane.
129. The method of clause 126, wherein the cancer cell membrane is a 3LL cell membrane.
130. The method of clause 126, wherein the cancer cell membrane is selected from the group consisting of a NCI-H1299 cell membrane, a HCC70 cell membrane, a RWPE-1 cell membrane, a CWR-R1 cell membrane, a C4-2 cell membrane, a HEK293 cell membrane, a PC-3 cell membrane, a SKOV3 cell membrane, a MDA PCa 2b cell membrane, a LNCaP95 cell membrane, a MCF-7 cell membrane, a SGBS cell membrane, a C4-2b cell membrane, a NHLF cell membrane, a LNCaP-C81 cell membrane, a Hela cell membrane, a VCaP cell membrane, a 293T cell membrane, a MDA-MB-468 cell membrane, a ATCC-231 (MDA-MB-231ATCC® HTB-26™) cell membrane, a ATCC-LNCaP (LNCaP clone FGCATCC® CRL-1740™) cell membrane, a RVE cell membrane, a LNCaP (LNCaP clone FGCATCC® CRL-1740™) cell membrane, a NDA157 (MDA-MB-157) cell membrane, and a PNT1A cell membrane.
131. The method of any one of clauses 86 to 121, wherein the cell membrane is an endothelial cell membrane.
132. The method of clause 131, wherein the endothelial cell membrane is a HUVEC cell membrane.
133. The method of clause 131, wherein the endothelial cell membrane is a human lung microvascular endothelial cell.
134. The method of any one of clauses 86 to 121, wherein the cell membrane is an epithelial cell membrane.
135. The method of any one of clauses 86 to 121, wherein the cell membrane is a bacterial cell membrane.
136. The method of clause 135, wherein the bacterial cell membrane is a gram-negative bacterial cell membrane.
137. The method of clause 136, wherein the gram-negative bacterial cell membrane is selected from the group consisting of E. coli, Neisseria gonorrhoeae, Chlamydia trachomatis, Yersinia pestis, Proteus, and Leptospiria.
138. The method of clause 135, wherein the bacterial cell membrane is a gram-positive bacterial cell membrane.
139. The method of clause 138, wherein the gram-positive bacterial cell membrane is selected from the group consisting of Mycoplasma, Bacillus, Staphylococcus, Streptomyces, and Enterococcus.
140. The method of clause 135, wherein the bacterial cell membrane is an Escherichia coli cell membrane.
141. The method of clause 135, wherein the bacterial cell membrane is a Pseudomonas cell membrane.
142. The method of any one of clauses 86 to 121, wherein the cell membrane is a viral cell membrane.
143. The method of any one of clauses 86 to 121, wherein the cell membrane is a primary cell membrane.
144. The method of any one of clauses 86 to 121, wherein the cell membrane is an immune cell membrane.
145. The method of any one of clauses 86 to 121, wherein the cell membrane is a human cell membrane.
146. The method of any one of clauses 86 to 121, wherein the cell membrane is a rodent cell membrane.
147. The method of any one of clauses 86 to 146, wherein the cell membrane forms the majority of the vesicle composition.
148. The method of any one of clauses 86 to 121, wherein the cell membrane is more than 50% of the vesicle composition by weight.
149. The method of any one of clauses 86 to 121, wherein the cell membrane is about 50% of the vesicle composition by weight.
150. The method of any one of clauses 86 to 121, wherein the cell membrane is about 75% of the vesicle composition by weight.
151. The method of any one of clauses 86 to 121, wherein the cell membrane is between 50%-75% of the vesicle composition by weight.
152. The method of any one of clauses 86 to 151, wherein the vesicle composition is formed via nitrogen cavitation.
153. The method of any one of clauses 86 to 152, wherein the vesicle composition is a cell-targeted vesicle composition.
154. The method of clause 153, wherein the cell-targeting corresponds to the cell type of the cell membrane.
155. The method of any one of clauses 86 to 154, wherein the agent is a therapeutic agent.
156. The method of clause 155, wherein the therapeutic agent is acidic.
157. The method of clause 155, wherein the therapeutic agent is basic.
158. The method of any one of clauses 155 to 157, wherein the therapeutic agent is an antibiotic.
159. The method of any one of clauses 155 to 157, wherein the therapeutic agent is an anti-inflammatory agent.
160. The method of clause 159, wherein the anti-inflammation agent is selected from the group consisting of anti-inflammatory glucocorticoids, NF-kB inhibitors, p38MAP kinase inhibitors, Syk/Zap kinase inhibitors, and siRNA oligonucleotides against genes involved in pro-inflammation.
161. The method of clause 159, wherein the anti-inflammation agent is selected from the group consisting of anti-inflammatory glucocorticoids, NF-kB inhibitors, p38MAP kinase inhibitors, Syk/Zap kinase inhibitors, and siRNA oligonucleotides against genes involved in pro-inflammation.
162. The method of clause 159, wherein the anti-inflammation agent is selected from the group consisting of TPCA-1 (2-[(Aminocarbonyl)amino]-5-(4-fluorophenyl)-3-thiophenecarboxamide), PS-1145 (N-(6-Chloro-9H-pyrido[3,4-b]indol-8-yl)-3-pyridinecarboxamide dihydrochloride), ML-120B (N-(6-Chloro-7-methoxy-9H-pyrido[3,4-b]indol-8-yl)-2-methyl-3-pyridinecarboxamide), SC-514 (4-Amino-[2′,3′-bithiophene]-5-carboxamide), IMD-0354 (N-[3,5-Bis(trifluoromethyl)phenyl]-5-chloro-2-hydroxybenzamide), BMS-345541 (N-(1,8-Dimethylimidazo[1,2-a]quinoxalin-4-yl)-1,2-ethanediamine hydrochloride), and Bay 11-7085 ((E)-3-(4-t-Butylphenylsulfonyl)-2-propenenitrile).
163. The method of clause 159, wherein the anti-inflammation agent is an anti-inflammatory glucocorticoid.
164. The method of clause 159, wherein the anti-inflammation agent is an NF-kB inhibitor.
165. The method of clause 159, wherein the anti-inflammation agent is a p38MAP kinase inhibitor.
166. The method of clause 159, wherein the anti-inflammation agent is a Syk/Zap kinase inhibitor.
167. The method of clause 159, wherein the anti-inflammation agent is an siRNA oligonucleotide against genes involved in pro-inflammation.
168. The method of clause 159, wherein the anti-inflammatory agent is piceatannol.
169. The method of any one of clauses 155 to 157, wherein the therapeutic agent is an anti-cancer agent.
170. The method of any one of clauses 155 to 157, wherein the therapeutic agent is an NF-κB inhibitor.
171. The method of any one of clauses 155 to 157, wherein the NF-κB inhibitor is TPCA-1.
172. A method of identifying a disorder in a patient, said method comprising the step of administering a vesicle composition comprising a cell membrane and an agent to the patient, wherein the administration of the vesicle composition identifies the disorder in the patient.
173. The method of clause 172, wherein the disorder is an inflammatory disorder.
174. The method of clause 173, wherein the inflammatory disorder is an acute inflammatory disease.
175. The method of clause 173, wherein the inflammatory disorder is a chronic inflammatory disease.
176. The method of clause 173, wherein the inflammatory disorder is cancer.
177. The method of clause 173, wherein the inflammatory disorder is sepsis.
178. The method of clause 173, wherein the inflammatory disorder is a lung injury.
179. The method of clause 178, wherein the lung injury is an acute lung injury.
180. The method of clause 178, wherein the lung injury is a chronic lung injury.
181. The method of clause 172, wherein the disorder is an infection.
182. The method of clause 181, wherein the infection is a bacterial infection.
183. The method of clause 181, wherein the infection is a viral infection.
184. The method of clause 181, wherein the infection is a fungal infection.
185. The method of any one of clauses 172 to 184, wherein the administration is a parenteral administration.
186. The method of clause 185, wherein the parenteral administration is an intravenous administration.
187. The method of any one of clauses 172 to 186, wherein the vesicle composition further comprises a targeting molecule.
188. The method of clause 187, wherein the targeting molecule is an intact targeting molecule.
189. The method of clause 187 or clause 188, wherein the targeting molecule is a membrane molecule.
190. The method of any one of clauses 187 to 189, wherein the targeting molecule is on the surface of the vesicle composition.
191. The method of any one of clauses 187 to 190, wherein the targeting molecule is derived from the cell membrane.
192. The method of any one of clauses 187 to 191, wherein the targeting molecule is a cell adhesion molecule.
193. The method of clause 192, wherein the cell adhesion molecule is an intercellular adhesion molecule.
194. The method of clause 192, wherein the cell adhesion molecule is integrin β2.
195. The method of clause 192, wherein the cell adhesion molecule is ICAM-1.
196. The method of any one of clauses 172 to 195, wherein the vesicle composition is a nanovesicle.
197. The method of any one of clauses 172 to 196, wherein the vesicle composition has an average diameter from about 40 nm to about 500 nm.
198. The method of any one of clauses 172 to 196, wherein the vesicle composition has an average diameter from about 100 nm to about 300 nm.
199. The method of any one of clauses 172 to 196, wherein the vesicle composition has an average diameter from about 80 nm to about 200 nm.
200. The method of any one of clauses 172 to 196, wherein the vesicle composition has an average diameter of about 100 nm.
201. The method of any one of clauses 172 to 196, wherein the vesicle composition has an average diameter of about 200 nm.
202. The method of any one of clauses 172 to 196, wherein the vesicle composition has an average diameter of about 300 nm.
203. The method of any one of clauses 172 to 196, wherein the vesicle composition has an average diameter of about 400 nm.
204. The method of any one of clauses 172 to 196, wherein the vesicle composition has an average diameter of about 500 nm.
205. The method of any one of clauses 172 to 204, wherein the vesicle composition is substantially free of one or more intracellular organelles.
206. The method of clause 205, wherein the intracellular organelles comprise one or more of endoplasmic reticulum, mitochondria, lysosomes, and Golgi bodies.
207. The method of any one of clauses 172 to 206, wherein the cell membrane is a neutrophil cell membrane.
208. The method of clause 207, wherein the neutrophil cell membrane is a HL60 cell membrane.
209. The method of clause 207, wherein the neutrophil cell membrane is a human neutrophil cell membrane.
210. The method of clause 207, wherein the neutrophil cell membrane is a rodent neutrophil cell membrane.
211. The method of any one of clauses 172 to 206, wherein the cell membrane is a cancer cell membrane.
212. The method of clause 211, wherein the cancer cell membrane is a HL60 cell membrane.
213. The method of clause 211, wherein the cancer cell membrane is a HeLa cell membrane.
214. The method of clause 211, wherein the cancer cell membrane is a 3LL cell membrane.
215. The method of clause 211, wherein the cancer cell membrane is selected from the group consisting of a NCI-H1299 cell membrane, a HCC70 cell membrane, a RWPE-1 cell membrane, a CWR-R1 cell membrane, a C4-2 cell membrane, a HEK293 cell membrane, a PC-3 cell membrane, a SKOV3 cell membrane, a MDA PCa 2b cell membrane, a LNCaP95 cell membrane, a MCF-7 cell membrane, a SGBS cell membrane, a C4-2b cell membrane, a NHLF cell membrane, a LNCaP-C81 cell membrane, a Hela cell membrane, a VCaP cell membrane, a 293T cell membrane, a MDA-MB-468 cell membrane, a ATCC-231 (MDA-MB-231ATCC® HTB-26™) cell membrane, a ATCC-LNCaP (LNCaP clone FGCATCC® CRL-1740™) cell membrane, a RVE cell membrane, a LNCaP (LNCaP clone FGCATCC® CRL-1740™) cell membrane, a NDA157 (MDA-MB-157) cell membrane, and a PNT1A cell membrane.
216. The method of any one of clauses 172 to 206, wherein the cell membrane is an endothelial cell membrane.
217. The method of clause 216, wherein the endothelial cell membrane is a HUVEC cell membrane.
218. The method of clause 216, wherein the endothelial cell membrane is a human lung microvascular endothelial cell.
219. The method of any one of clauses 172 to 206, wherein the cell membrane is an epithelial cell membrane.
220. The method of any one of clauses 172 to 206, wherein the cell membrane is a bacterial cell membrane.
221. The method of clause 220, wherein the bacterial cell membrane is a gram-negative bacterial cell membrane.
222. The method of clause 221, wherein the gram-negative bacterial cell membrane is selected from the group consisting of E. coli, Neisseria gonorrhoeae, Chlamydia trachomatis, Yersinia pestis, Proteus, and Leptospiria.
223. The method of clause 220, wherein the bacterial cell membrane is a gram-positive bacterial cell membrane.
224. The method of clause 223, wherein the gram-positive bacterial cell membrane is selected from the group consisting of Mycoplasma, Bacillus, Staphylococcus, Streptomyces, and Enterococcus.
225. The method of clause 220, wherein the bacterial cell membrane is an Escherichia coli cell membrane.
226. The method of clause 220, wherein the bacterial cell membrane is a Pseudomonas cell membrane.
227. The method of any one of clauses 172 to 206, wherein the cell membrane is a viral cell membrane.
228. The method of any one of clauses 172 to 206, wherein the cell membrane is a primary cell membrane.
229. The method of any one of clauses 172 to 206, wherein the cell membrane is an immune cell membrane.
230. The method of any one of clauses 172 to 206, wherein the cell membrane is a human cell membrane.
231. The method of any one of clauses 172 to 206, wherein the cell membrane is a rodent cell membrane.
232. The method of any one of clauses 172 to 231, wherein the cell membrane forms the majority of the vesicle composition.
233. The method of any one of clauses 172 to 231, wherein the cell membrane is more than 50% of the vesicle composition by weight.
234. The method of any one of clauses 172 to 231, wherein the cell membrane is about 50% of the vesicle composition by weight.
235. The method of any one of clauses 172 to 231, wherein the cell membrane is about 75% of the vesicle composition by weight.
236. The method of any one of clauses 172 to 231, wherein the cell membrane is between 50%-75% of the vesicle composition by weight.
237. The method of any one of clauses 172 to 236, wherein the vesicle composition is formed via nitrogen cavitation.
238. The method of any one of clauses 172 to 237, wherein the vesicle composition is a cell-targeted vesicle composition.
239. The method of clause 238, wherein the cell-targeting corresponds to the cell type of the cell membrane.
240. The method of any one of clauses 172 to 239, wherein the agent is a diagnostic agent.
241. The method of clause 240, wherein the diagnostic agent is acidic.
242. The method of clause 240, wherein the diagnostic agent is basic.
243. The method of any one of clauses 240 to 242, wherein the diagnostic agent is selected from the group consisting of fluorescent probes or tags, isotope probes or tags, antibody probes or tags, antigen probes or tags, enzyme probes or tags, dye probes or tags, biotin-binding protein probes or tags, and bioluminescence reporter probes or tags.
244. The method of any one of clauses 240 to 242, wherein the diagnostic agent is a fluorescent probe or tag.
245. The method of any one of clauses 240 to 242, wherein the diagnostic agent is an isotope probe or tag.
246. The method of any one of clauses 240 to 242, wherein the diagnostic agent is an antibody probe or tag.
247. The method of any one of clauses 240 to 242, wherein the diagnostic agent is an antigen probe or tag.
248. The method of any one of clauses 240 to 242, wherein the diagnostic agent is an enzyme probe or tag.
249. The method of any one of clauses 240 to 242, wherein the diagnostic agent is a dye probe or tag.
250. The method of any one of clauses 240 to 242, wherein the diagnostic agent is a biotin-binding protein probe or tag.
251. The method of any one of clauses 240 to 242, wherein the diagnostic agent is a bioluminescence reporter probe or tag.
252. The method of any one of clauses 240 to 242, wherein the diagnostic agent is a photosensitizer.
253. The method of clause 252, wherein the photosensitizer is a derivative of porphyrin and/or chlorophyli.
254. The method of clause 252, wherein the photosensitizer is selected from the group consisting of Allumera, Photofrin,Visudyne, Levulan, Foscan, Metvix, Hexvix, Cysview, and Laserphyrin.
255. A method of making a vesicle composition comprising a cell membrane, said method comprising the steps of:
(a)performing nitrogen cavitation on a cell to provide a solution comprising membrane vesicles;
(b) centrifuging the solution one or more times to provide a centrifuged product; and
(c) extruding the centrifuged product through a porous membrane to provide the vesicle composition.
256. The method of clause 255, wherein at least one instance of the centrifuging step comprises centrifuging the solution at about 2000 g.
257. The method of clause 255 or clause 256, wherein at least one instance of the centrifuging step comprises centrifuging the solution at about 100,000 g.
258. The method of any one of clauses 255 to 257, wherein the method further comprises the step of sonicating the vesicle composition.
259. The method of clause 258, wherein the sonication of the vesicle composition changes the pH inside of the vesicle composition.
260. The method of clause 259, wherein the pH inside of the vesicle composition increases as a result of sonication.
261. The method of clause 259, wherein the pH inside of the vesicle composition decreases as a result of sonication.
262. The method of any one of clauses 255 to 261, wherein the method further comprises the step of loading an agent into the vesicle composition.
263. The method of clause 262, wherein the loading of the agent into the vesicle composition is performed subsequent to the step of sonicating the vesicle composition.
264. The method of clause 262 or clause 263, wherein the loading of the agent into the vesicle composition is performed via incubation of the agent with the vesicle composition.
265. The method of any one of clauses 262 to 264, wherein the loading efficiency of the agent into the vesicle composition is improved by i) increasing or decreasing the pH value inside of the vesicle composition and/or ii) increasing or decreasing the pH value outside of the vehicle composition.
266. The method of any one of clauses 262 to 264, wherein the loading efficiency of the agent into the vesicle composition is improved by a pH gradient between the inside of and the outside of the vesicle composition.
267. The method of any one of clauses 262 to 266, wherein the agent is a therapeutic agent.
268. The method of clause 267, wherein the therapeutic agent is acidic.
269. The method of clause 267, wherein the therapeutic agent is basic.
270. The method of any one of clauses 267 to 269, wherein the therapeutic agent is an antibiotic.
271. The method of any one of clauses 267 to 269, wherein the therapeutic agent is an anti-inflammatory agent.
272. The method of clause 271, wherein the anti-inflammation agent is selected from the group consisting of anti-inflammatory glucocorticoids, NF-kB inhibitors, p38MAP kinase inhibitors, Syk/Zap kinase inhibitors, and siRNA oligonucleotides against genes involved in pro-inflammation.
273. The method of clause 271, wherein the anti-inflammation agent is selected from the group consisting of anti-inflammatory glucocorticoids, NF-kB inhibitors, p38MAP kinase inhibitors, Syk/Zap kinase inhibitors, and siRNA oligonucleotides against genes involved in pro-inflammation.
274. The method of clause 271, wherein the anti-inflammation agent is selected from the group consisting of TPCA-1 (2-[(Aminocarbonyl)amino]-5-(4-fluorophenyl)-3-thiophenecarboxamide), PS-1145 (N-(6-Chloro-9H-pyrido[3,4-b]indol-8-yl)-3-pyridinecarboxamide dihydrochloride), ML-120B (N-(6-Chloro-7-methoxy-9H-pyrido[3,4-b]indol-8-yl)-2-methyl-3-pyridinecarboxamide), SC-514 (4-Amino-[2′,3′-bithiophene]-5-carboxamide), IMD-0354 (N-[3,5-Bis(trifluoromethyl)phenyl]-5-chloro-2-hydroxybenzamide), BMS-345541 (N-(1,8-Dimethylimidazo[1,2-a]quinoxalin-4-yl)-1,2-ethanediamine hydrochloride), and Bay 11-7085 ((E)-3-(4-t-Butylphenylsulfonyl)-2-propenenitrile).
275. The method of clause 271, wherein the anti-inflammation agent is an anti-inflammatory glucocorticoid.
276. The method of clause 271, wherein the anti-inflammation agent is an NF-kB inhibitor.
277. The method of clause 271, wherein the anti-inflammation agent is a p38MAP kinase inhibitor.
278. The method of clause 271, wherein the anti-inflammation agent is a Syk/Zap kinase inhibitor.
279. The method of clause 271, wherein the anti-inflammation agent is an siRNA oligonucleotide against genes involved in pro-inflammation.
280. wherein the anti-inflammatory agent is piceatannol.
281. The method of any one of clauses 267 to 269, wherein the therapeutic agent is an anti-cancer agent.
282. The method of any one of clauses 267 to 269, wherein the therapeutic agent is an NF-κB inhibitor.
283. The method of clause 282, wherein the NF-κB inhibitor is TPCA-1.
284. The method of any one of clauses 262 to 266, wherein the agent is a diagnostic agent.
285. The method of clause 284, wherein the diagnostic agent is acidic.
286. The method of clause 284, wherein the diagnostic agent is basic.
287. The method of any one of clauses 284 to 286, wherein the diagnostic agent is selected from the group consisting of fluorescent probes or tags, isotope probes or tags, antibody probes or tags, antigen probes or tags, enzyme probes or tags, dye probes or tags, biotin-binding protein probes or tags, and bioluminescence reporter probes or tags.
288. The method of any one of clauses 284 to 286, wherein the diagnostic agent is a fluorescent probe or tag.
289. The method of any one of clauses 284 to 286, wherein the diagnostic agent is an isotope probe or tag.
290. The method of any one of clauses 284 to 286, wherein the diagnostic agent is an antibody probe or tag.
291. The method of any one of clauses 284 to 286, wherein the diagnostic agent is an antigen probe or tag.
292. The method of any one of clauses 284 to 286, wherein the diagnostic agent is an enzyme probe or tag.
293. The method of any one of clauses 284 to 286, wherein the diagnostic agent is a dye probe or tag.
294. The method of any one of clauses 284 to 286, wherein the diagnostic agent is a biotin-binding protein probe or tag.
295. The method of any one of clauses 284 to 286, wherein the diagnostic agent is a bioluminescence reporter probe or tag.
296. The method of any one of clauses 255 to 295, wherein the vesicle composition further comprises a targeting molecule
297. The method of clause 296, wherein the targeting molecule is an intact targeting molecule.
298. The method of clause 296 or clause 297, wherein the targeting molecule is a membrane molecule.
299. The method of any one of clauses 296 to 298, wherein the targeting molecule is on the surface of the vesicle composition.
300. The method of any one of clauses 296 to 299, wherein the targeting molecule is derived from the cell membrane.
301. The method of any one of clauses 296 to 300, wherein the targeting molecule is a cell adhesion molecule.
302. The method of clause 301, wherein the cell adhesion molecule is an intercellular adhesion molecule.
303. The method of clause 301, wherein the cell adhesion molecule is integrin β2.
304. The method of clause 301, wherein the cell adhesion molecule is ICAM-1.
305. The method of any one of clauses 255 to 304, wherein the vesicle composition is a nanovesicle.
306. The method of any one of clauses 255 to 304, wherein the vesicle composition has an average diameter from about 40 nm to about 500 nm.
307. The method of any one of clauses 255 to 304, wherein the vesicle composition has an average diameter from about 100 nm to about 300 nm.
308. The method of any one of clauses 255 to 304, wherein the vesicle composition has an average diameter from about 80 nm to about 200 nm.
309. The method of any one of clauses 255 to 304, wherein the vesicle composition has an average diameter of about 100 nm.
310. The method of any one of clauses 255 to 304, wherein the vesicle composition has an average diameter of about 200 nm.
311. The method of any one of clauses 255 to 304, wherein the vesicle composition has an average diameter of about 300 nm.
312. The method of any one of clauses 255 to 304, wherein the vesicle composition has an average diameter of about 400 nm.
313. The method of any one of clauses 255 to 304, wherein the vesicle composition has an average diameter of about 500 nm.
314. The method of any one of clauses 255 to 313, wherein the vesicle composition is substantially free of one or more intracellular organelles.
315. The method of clause 314, wherein the intracellular organelles comprise one or more of endoplasmic reticulum, mitochondria, lysosomes, and Golgi bodies.
316. The method of any one of clauses 255 to 315, wherein the cell membrane is a neutrophil cell membrane.
317. The method of clause 316, wherein the neutrophil cell membrane is a HL60 cell membrane.
318. The method of clause 316, wherein the neutrophil cell membrane is a human neutrophil cell membrane.
319. The method of clause 316, wherein the neutrophil cell membrane is a rodent neutrophil cell membrane.
320. The method of any one of clauses 255 to 315, wherein the cell membrane is a cancer cell membrane.
321. The method of clause 320, wherein the cancer cell membrane is a HL60 cell membrane.
322. The method of clause 320, wherein the cancer cell membrane is a HeLa cell membrane.
323. The method of clause 320, wherein the cancer cell membrane is a 3LL cell membrane.
324. The method of clause 320, wherein the cancer cell membrane is selected from the group consisting of a NCI-H1299 cell membrane, a HCC70 cell membrane, a RWPE-1 cell membrane, a CWR-R1 cell membrane, a C4-2 cell membrane, a HEK293 cell membrane, a PC-3 cell membrane, a SKOV3 cell membrane, a MDA PCa 2b cell membrane, a LNCaP95 cell membrane, a MCF-7 cell membrane, a SGBS cell membrane, a C4-2b cell membrane, a NHLF cell membrane, a LNCaP-C81 cell membrane, a Hela cell membrane, a VCaP cell membrane, a 293T cell membrane, a MDA-MB-468 cell membrane, a ATCC-231 (MDA-MB-231ATCC® HTB-26™) cell membrane, a ATCC-LNCaP (LNCaP clone FGCATCC® CRL-1740™) cell membrane, a RVE cell membrane, a LNCaP (LNCaP clone FGCATCC® CRL-1740™) cell membrane, a NDA157 (MDA-MB-157) cell membrane, and a PNT1A cell membrane.
325. The method of any one of clauses 255 to 315, wherein the cell membrane is an endothelial cell membrane.
326. The method of clause 325, wherein the endothelial cell membrane is a HUVEC cell membrane.
327. The method of clause 325, wherein the endothelial cell membrane is a human lung microvascular endothelial cell.
328. The method of any one of clauses 255 to 315, wherein the cell membrane is an epithelial cell membrane.
329. The method of any one of clauses 255 to 315, wherein the cell membrane is a bacterial cell membrane.
330. The method of clause 329, wherein the bacterial cell membrane is a gram-negative bacterial cell membrane.
331. The method of clause 330, wherein the gram-negative bacterial cell membrane is selected from the group consisting of E. coli, Neisseria gonorrhoeae, Chlamydia trachomatis, Yersinia pestis, Proteus, and Leptospiria.
332. The method of clause 329, wherein the bacterial cell membrane is a gram-positive bacterial cell membrane.
333. The method of clause 332, wherein the gram-positive bacterial cell membrane is selected from the group consisting of Mycoplasma, Bacillus, Staphylococcus, Streptomyces, and Enterococcus.
334. The method of clause 329, wherein the bacterial cell membrane is an Escherichia coli cell membrane.
335. The method of clause 329, wherein the bacterial cell membrane is a Pseudomonas cell membrane.
336. The method of any one of clauses 255 to 315, wherein the cell membrane is a viral cell membrane.
337. The method of any one of clauses 255 to 315, wherein the cell membrane is a primary cell membrane.
338. The method of any one of clauses 255 to 315, wherein the cell membrane is an immune cell membrane.
339. The method of any one of clauses 255 to 315, wherein the cell membrane is a human cell membrane.
340. The method of any one of clauses 255 to 315, wherein the cell membrane is a rodent cell membrane.
341. The method of any one of clauses 255 to 340, wherein the cell membrane forms the majority of the vesicle composition.
342. The method of any one of clauses 255 to 340, wherein the cell membrane is more than 50% of the vesicle composition by weight.
343. The method of any one of clauses 255 to 340, wherein the cell membrane is about 50% of the vesicle composition by weight.
344. The method of any one of clauses 255 to 340, wherein the cell membrane is about 75% of the vesicle composition by weight.
345. The method of any one of clauses 255 to 340, wherein the cell membrane is between 50%-75% of the vesicle composition by weight.
346. The method of any one of clauses 255 to 345, wherein the vesicle composition is formed via nitrogen cavitation.
347. The method of any one of clauses 255 to 346, wherein the vesicle composition is a cell-targeted vesicle composition.
348. The method of clause 347, wherein the cell-targeting corresponds to the cell type of the cell membrane.

### DESCRIPTION

Various embodiments of the invention are described herein as follows. In one aspect of the present disclosure, a vesicle composition is provided. The vesicle composition comprises a cell membrane and an agent. In another aspect of the present disclosure, a method of treating a disease in a patient in need thereof is provided. The method comprises the step of administering a vesicle composition comprising a cell membrane and an agent to the patient, wherein the administration of the vesicle composition reduces one or more symptoms associated with the disease. In yet another aspect of the present disclosure, a method of identifying a disorder in a patient is provided. The method comprises the step of administering a vesicle composition comprising a cell membrane and an agent to the patient, wherein the administration of the vesicle composition identifies the disorder in the patient. In another aspect of the present disclosure, a method of making a vesicle composition comprising a cell membrane is provided. The method comprises the steps of (a) performing nitrogen cavitation on a cell to provide a solution comprising membrane vesicles; (b) centrifuging the solution one or more times to provide a centrifuged product; and (c) extruding the centrifuged product through a porous membrane to provide the vesicle composition.

In one aspect of the present disclosure, a vesicle composition is provided. The vesicle composition comprises a cell membrane and an agent. In certain aspects, the vesicle composition further comprises a targeting molecule. The targeting molecule can be utilized to target the vesicle composition to a particular cell type or cell types in a patient, for example if the vesicle composition comprises a cell membrane of the particular cell type or cell types.

In some embodiments, the targeting molecule is an intact targeting molecule. In certain embodiments, the targeting molecule is a membrane molecule, for example a cell membrane molecule. In other embodiments, the targeting molecule is on the surface of the vesicle composition. In various embodiments, the targeting molecule is derived from the cell membrane.

In some embodiments, the targeting molecule is a cell adhesion molecule. In various embodiments, the cell adhesion molecule is an intercellular adhesion molecule. In other embodiments, the cell adhesion molecule is integrin β2. In yet other embodiments, the cell adhesion molecule is Intercellular Adhesion Molecule 1 (ICAM-1), also known as CD54 (Cluster of Differentiation 54).

In certain aspects, the vesicle composition is a nanovesicle. In some embodiments, the vesicle composition has an average diameter from about 40 nm to about 500 nm. In various embodiments, the vesicle composition has an average diameter from about 100 nm to about 300 nm. In certain embodiments, the vesicle composition has an average diameter from about 80 nm to about 200 nm. In one embodiment, the vesicle composition has an average diameter of about 100 nm. In another embodiment, the vesicle composition has an average diameter of about 200 nm. In yet another embodiment, the vesicle composition has an average diameter of about 300 nm. In yet another embodiment, the vesicle composition has an average diameter of about 400 nm. In yet another embodiment, the vesicle composition has an average diameter of about 500 nm.

In certain embodiments, the vesicle composition is substantially free of one or more intracellular organelles. As used herein, the term “substantially free” includes a non-appreciable amount of one or more intracellular organelles that may be present in a composition of the present invention. In some embodiments, the intracellular organelles comprise one or more of endoplasmic reticulum, mitochondria, lysosomes, and Golgi bodies. For example, in one aspect, the intracellular organelles comprise endoplasmic reticulum. In another aspect, the intracellular organelles comprise mitochondria. In another aspect, the intracellular organelles comprise lysosomes. In yet another aspect, the intracellular organelles comprise Golgi bodies.

In various embodiments, the cell membrane is a neutrophil cell membrane. In some embodiments, the neutrophil cell membrane is a HL60 cell membrane. In other embodiments, the neutrophil cell membrane is a human neutrophil cell membrane. In yet other embodiments, the neutrophil cell membrane is a rodent neutrophil cell membrane.

In various embodiments, the cell membrane is a cancer cell membrane. In some embodiments, the cancer cell membrane is a HL60 cell membrane. In other embodiments, the cancer cell membrane is a HeLa cell membrane. In other embodiments, the cancer cell membrane is a 3LL cell membrane. In yet other embodiments, the cancer cell membrane is selected from the group consisting of a NCI-H1299 cell membrane, a HCC70 cell membrane, a RWPE-1 cell membrane, a CWR-R1 cell membrane, a C4-2 cell membrane, a HEK293 cell membrane, a PC-3 cell membrane, a SKOV3 cell membrane, a MDA PCa 2b cell membrane, a LNCaP95 cell membrane, a MCF-7 cell membrane, a SGBS cell membrane, a C4-2b cell membrane, a NHLF cell membrane, a LNCaP-C81 cell membrane, a Hela cell membrane, a VCaP cell membrane, a 293T cell membrane, a MDA-MB-468 cell membrane, a ATCC-231 (MDA-MB-231ATCC® HTB-26™) cell membrane, a ATCC-LNCaP (LNCaP clone FGCATCC® CRL-1740™) cell membrane, a RVE cell membrane, a LNCaP (LNCaP clone FGCATCC® CRL-1740™) cell membrane, a NDA157 (MDA-MB-157) cell membrane, and a PNT1A cell membrane. The various cell types are known as follows: NCI-H1299 (non-small cell lung cancer), HCC70 (primary ductal carcinoma), RWPE-1 (prostate normal), CWR-R1 (prostate carcicinoma), C4-2 (prostate, carcinoma); HEK293 (embryonic kidney); PC3 (prostate adenocarcinoma); SKOV3 (ovary adenocarcinoma); MDA PCa 2b (prostate adenocarcinoma); LNCaP95 (prostate cancer); MCF-7 (breast adenocarcinoma; SGBS (preadipocyte); C4-2b (prostate, carcinoma); NHLF (Normal Human lung fibroblasts); LNCaP-C81 (prostate, carcinoma) Hela (cervix adenocarcinoma; VCaP (prostate cancer); 293T (embryonic kidney); MDA-MB-468 (breast adenocarcinoma); ATCC-231 (MDA-MB-231ATCC® HTB-26™) (breast adenocarcinoma); ATCC-LNCaP (LNCaP clone FGCATCC® CRL-1740™) (prostate cancer); RVE (prostate, carcinoma); LNCaP (LNCaP clone FGCATCC® CRL-1740™) (prostate cancer); NDA157 (MDA-MB-157) (breast carcinoma); PNT1A (human Normal prostate epithelium).

In various embodiments, the cell membrane is an endothelial cell membrane. In some embodiments, the endothelial cell membrane is a human umbilical vein endothelial cell (HUVEC) cell membrane. In other embodiments, the endothelial cell membrane is a human lung microvascular endothelial cell. In other various embodiments, the cell membrane is an epithelial cell membrane.

In various embodiments, the cell membrane is a bacterial cell membrane. In certain aspects, the bacterial cell membrane is a gram-negative bacterial cell membrane. In some embodiments, the gram-negative bacterial cell membrane is selected from the group consisting of E. coli, Neisseria gonorrhoeae, Chlamydia trachomatis, Yersinia pestis, Proteus, and Leptospiria. In other aspects, the bacterial cell membrane is a gram-positive bacterial cell membrane. In some embodiments, the gram-positive bacterial cell membrane is selected from the group consisting of Mycoplasma, Bacillus, Staphylococcus, Streptomyces, and Enterococcus. In one embodiment, the bacterial cell membrane is an Escherichia coli cell membrane. In another embodiment, the bacterial cell membrane is a Pseudomonas cell membrane.

In one aspect, the cell membrane is a viral cell membrane. In another aspect, the cell membrane is a primary cell membrane. In yet another aspect, the cell membrane is an immune cell membrane. In another aspect, the cell membrane is a human cell membrane. In yet another aspect, the cell membrane is a rodent cell membrane.

In certain embodiments, the cell membrane forms the majority of the vesicle composition. In one embodiment, the cell membrane is more than 50% of the vesicle composition by weight. In another embodiment, the cell membrane is about 50% of the vesicle composition by weight. In yet another embodiment, wherein the cell membrane is about 75% of the vesicle composition by weight. In certain aspects, the cell membrane is between 50%-75% of the vesicle composition by weight.

In some aspects, the vesicle composition is formed via nitrogen cavitation. Methods of performing nitrogen cavitation are described herein and are well known to the skilled artisan. In various embodiments, the vesicle composition is a cell-targeted vesicle composition. As used herein, a “cell-targeted vesicle composition” means that the vesicle composition preferentially targets a certain cell type or cell types within the body. For example, in some embodiments, the cell-targeting corresponds to the cell type of the cell membrane utilized in the vesicle composition.

In certain embodiments, the agent is a therapeutic agent. In some embodiments, the therapeutic agent is acidic. In other embodiments, the therapeutic agent is basic. In various aspects, the therapeutic agent is an antibiotic. In other aspects, the therapeutic agent is an anti-inflammatory agent. In some embodiments, the anti-inflammation agent is selected from the group consisting of anti-inflammatory glucocorticoids, NF-kB inhibitors, p38MAP kinase inhibitors, Syk/Zap kinase inhibitors, and siRNA oligonucleotides against genes involved in pro-inflammation. In other embodiments, the anti-inflammation agent is selected from the group consisting of TPCA-1 (2-[(Aminocarbonyl)amino]-5-(4-fluorophenyl)-3-thiophenecarboxamide), PS-1145 (N-(6-Chloro-9H-pyrido[3,4-b]indol-8-yl)-3-pyridinecarboxamide dihydrochloride), ML-120B (N-(6-Chloro-7-methoxy-9H-pyrido[3,4-b]indol-8-yl)-2-methyl-3-pyridinecarboxamide), SC-514 (4-Amino-[2′,3′-bithiophene]-5-carboxamide), IMD-0354 (N-[3,5-Bis(trifluoromethyl)phenyl]-5-chloro-2-hydroxybenzamide), BMS-345541 (N-(1,8-Dimethylimidazo[1,2-a]quinoxalin-4-yl)-1,2-ethanediamine hydrochloride), and Bay 11-7085 ((E)-3-(4-t-Butylphenylsulfonyl)-2-propenenitrile). In yet other embodiments, the anti-inflammation agent is an anti-inflammatory glucocorticoid. In another embodiment, the anti-inflammation agent is an NF-kB inhibitor. In yet another embodiment, the anti-inflammation agent is a p38MAP kinase inhibitor. In another embodiment, the anti-inflammation agent is a Syk/Zap kinase inhibitor. In yet another embodiment, the anti-inflammation agent is an siRNA oligonucleotide against genes involved in pro-inflammation. In another embodiment, the anti-inflammatory agent is piceatannol.

In certain aspects, the therapeutic agent is an anti-cancer agent. In other aspects, the therapeutic agent is an NF-κB inhibitor. In some embodiments, the NF-κB inhibitor is TPCA-1.

In other aspects, the agent is a diagnostic agent. In some embodiments, the diagnostic agent is acidic. In other embodiments, the diagnostic agent is basic. In various embodiments, the diagnostic agent is selected from the group consisting of fluorescent probes or tags, isotope probes or tags, antibody probes or tags, antigen probes or tags, enzyme probes or tags, dye probes or tags, biotin-binding protein probes or tags, and bioluminescence reporter probes or tags. In one embodiment, the diagnostic agent is a fluorescent probe or tag. In another embodiment, the diagnostic agent is an isotope probe or tag. In yet another embodiment, the diagnostic agent is an antibody probe or tag. In another embodiment, the diagnostic agent is an antigen probe or tag. In yet another embodiment, the diagnostic agent is an enzyme probe or tag. In another embodiment, the diagnostic agent is a dye probe or tag. In another embodiment, the diagnostic agent is a biotin-binding protein probe or tag. In another embodiment, the diagnostic agent is a bioluminescence reporter probe or tag.

In certain aspects, the diagnostic agent is a photosensitizer. In some embodiments, the photosensitizer is a derivative of porphyrin and/or chlorophyli. In various aspects, the photosensitizer is selected from the group consisting of Allumera, Photofrin, Visudyne, Levulan, Foscan, Metvix, Hexvix, Cysview, and Laserphyrin.

In another aspect of the present disclosure, a method of treating a disease in a patient in need thereof is provided. The method comprises the step of administering a vesicle composition comprising a cell membrane and an agent to the patient, wherein the administration of the vesicle composition reduces one or more symptoms associated with the disease. The previously described embodiments of the vesicle composition are applicable to the method of treating a disease in a patient in need thereof described herein.

In various embodiments, the disease is an inflammatory disease. In some embodiments, the inflammatory disease is an acute inflammatory disease. In other embodiments, the inflammatory disease is a chronic inflammatory disease. In yet other embodiments, the inflammatory disease is cancer. In other embodiments, the inflammatory disease is sepsis.

In certain aspects, the inflammatory disease is a lung injury. In one aspect, the lung injury is an acute lung injury. In another aspect, the lung injury is a chronic lung injury.

In other aspects, the disease is an infection. In some embodiments, the infection is a bacterial infection. In other embodiments, the infection is a viral infection. In yet other embodiments, the infection is a fungal infection.

In certain embodiments, the administration is a parenteral administration. Suitable routes for such parenteral administration include intravenous, intraarterial, intraperitoneal, intrathecal, intradermal, epidural, intracerebroventricular, intraurethral, intrasternal, intracranial, intratumoral, intramuscular and subcutaneous delivery. In some embodiments, the parenteral administration is an intravenous administration. Suitable means for parenteral administration include needle (including microneedle) injectors, needle-free injectors and infusion techniques.

In other embodiments, the administration is an oral administration. The term “oral administration” refers to the provision of a composition via the mouth through ingestion, or via some other part of the gastrointestinal system including the esophagus. Examples of oral dosage forms include tablets (including compressed, coated or uncoated), capsules, hard or soft gelatin capsules, pellets, pills, powders, granules, elixirs, tinctures, colloidal dispersions, dispersions, effervescent compositions, films, sterile solutions or suspensions, syrups and emulsions and the like.

In certain aspects, a therapeutically effective amount of the vesicle composition is administered to the patient. As used herein, the term “therapeutically effective amount” refers to an amount which gives the desired benefit to a patient and includes both treatment and prophylactic administration. The amount will vary from one patient to another and will depend upon a number of factors, including the overall physical condition of the patient and the underlying cause of the condition to be treated. As used herein, the term “patient” refers to an animal, for example a human.

A “therapeutically effective amount” can be determined by a skilled artisan, and can be calculated based on the amount of drug in the vesicle composition, on the amount of albumin in the vesicle composition, or both. In some embodiments, the therapeutically effective amount of the vesicle composition is administered to the patient at a dose of about 0.001 to about 1000 mg. In one embodiment, the therapeutically effective amount of the vesicle composition is administered to the patient at a dose of about 0.001 to about 100 mg. In another embodiment, the therapeutically effective amount of the vesicle composition is administered to the patient at a dose of about 0.01 to about 100 mg. In yet another embodiment, the therapeutically effective amount of the vesicle composition is administered to the patient at a dose of about 0.1 to about 100 mg. In one embodiment, the therapeutically effective amount of the vesicle composition is administered to the patient at a dose of about 0.1 to about 10 mg. In one aspect of the described method, the disease is cancer and wherein the method is cancer immunotherapy.

In some embodiments, the administration reduces ICAM-1 expression in the patient. Methods of determining ICAM-1 expression in a patient are well known to the skilled artisan.

In yet another aspect of the present disclosure, a method of identifying a disorder in a patient is provided. The method comprises the step of administering a vesicle composition comprising a cell membrane and an agent to the patient, wherein the administration of the vesicle composition identifies the disorder in the patient. The previously described embodiments of the vesicle composition are applicable to the method of identifying a disorder in a patient in need thereof described herein. Furthermore, the previously described embodiments of treating a disease in a patient in need thereof are applicable to the method of identifying a disorder in a patient in need thereof described herein.

In another aspect of the present disclosure, a method of making a vesicle composition comprising a cell membrane is provided. The method comprises the steps of (a) performing nitrogen cavitation on a cell to provide a solution comprising membrane vesicles; (b) centrifuging the solution one or more times to provide a centrifuged product; and (c) extruding the centrifuged product through a porous membrane to provide the vesicle composition. The previously described embodiments of the vesicle composition are applicable to the method of making a vesicle composition described herein.

In certain embodiments, at least one instance of the centrifuging step comprises centrifuging the solution at about 2000 g. In other embodiments, at least one instance of the centrifuging step comprises centrifuging the solution at about 100,000 g. The method of making a vesicle composition described herein can comprise two or more centrifuging steps. For example, the method can comprise one centrifuging step at about 2000 g and a second centrifuging step at about 100,000 g. Furthermore, the method can comprise other centrifuging steps at speeds known to the skilled artisan. After the various centrifuging steps, the method may include lyophilization of the resulting pellet, weighing of the resulting pellet, quantification of materials comprising the resulting pellet, and/or modification of the resulting pellet.

In various aspects, the method further comprises the step of sonicating the vesicle composition. Methods of sonication are well known to the skilled artisan. In some aspects, the sonication of the vesicle composition changes the pH inside of the vesicle composition. In one aspect, the pH inside of the vesicle composition increases as a result of sonication. In another aspect, the pH inside of the vesicle composition decreases as a result of sonication.

In certain embodiments, the method further comprises the step of loading an agent into the vesicle composition. In some embodiments, the loading of the agent into the vesicle composition is performed subsequent to the step of sonicating the vesicle composition. In other embodiments, the loading of the agent into the vesicle composition is performed via incubation of the agent with the vesicle composition. In one embodiment, the loading efficiency of the agent into the vesicle composition is improved by i) increasing or decreasing the pH value inside of the vesicle composition and/or ii) increasing or decreasing the pH value outside of the vehicle composition. In another embodiment, the loading efficiency of the agent into the vesicle composition is improved by a pH gradient between the inside of and the outside of the vesicle composition. Methods of formulating the pH gradient between the inside of and the outside of the vesicle composition are known to the skilled artisan.

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

Examples 2-7 utilize the following exemplary materials and methods.

Reagents and Chemicals: LPS (Escherichia coli 0111:B4), formaldehyde solution and dimethyl sulfoxide (DMSO, purity>99.5%) were obtained from Sigma-Aldrich (St. Louis, Mo.). Recombinant human and mouse TNF-α (10 μg, carrier-free, purity>98%), Alexa Fluor@647 anti-mouse CD31 antibody, Alexa Fluor@488 anti-mouse Gr-1 (Ly-6G/Ly-6C) antibody and ELISA kits for TNF-α and IL-6 were purchased from Biolegend (San Diego, Calif.). Human HL60 cell lines were obtained from ATCC (Manassas, Va.) and erythrocytes were purchased from Zen-Bio (Research Triangle Park, N.C.). Anti-ICAM-1 antibodies and anti-Integrin-β₂ antibodies were purchased from Santa Cruz Biotechnogies (Santa Cruz, Calif.). TPCA-1 was purchased from Tocris Bioscience (Minneapolis, Minn.). Human vascular endothelial cells (HUVECs) were obtained from Lonza (Walkersville, Md.). Formavar carbon film on 100 mesh nickel grid for TEM was obtained from Electron Microscopy Sciences. Diff-Quick dye was purchased from Polysciences Inc. (Warrington, Pa.). DiO (Benzoxazolium, 3-octadecyl-2-[3-(3-octadecyl-2(3H)-benzoxazolylidene)-1-propenyl]-, perchlorate) [Ex(484 nm)\Em(501 nm)] and Dil (3H-Indolium, 2-(3-(1,3-dihydro-3,3-dimethyl-1-octadecyl-2H-indol-2-ylidene)-1-propenyl)-3,3-dimethyl-1-octadecyl-, perchlorate) [(Ex(549 nm)\Em(565 nm)], DAPI, pen strep, glutamine (100×) were purchased from Life Technologies, Grand Island, N.Y. QuantiFluor dsDNA detection kit was purchased from Promega. Pierce™ BCA protein assay kit was purchased from ThermoFisher Scientific.

HL60 cell culture and their activation: Human HL 60 cells were cultured in RPMI1640 (Lonza, Walkersville, Md.) supplemented with 10% (v/v) FBS (Seradigm, Radnor, Pa.) and 1% (v/v) pen strep glutamine. To activate HL 60 cells to express integrin β₂, 1.3% (v./v.) DMSO was added into the culture medium and the cells were cultured for about 4 to 6 days. Integrin β₂ expression was determined by Western blot.

Preparation of cell-derived vesicles, their drug loading and fluorescently labeling: DMSO-treated HL60 cells were harvested and washed with HBSS (without Ca²⁺, Mg²⁺ and phenol red, Corning, Inc., Corning, N.Y.). The cells were re-suspended in HBSS at a concentration of 1-5×10⁷/ml. Approximately 10 ml of suspension with 2.5 mM MgCl₂ was placed in a nitrogen cavitation vessel (Parr instrument, Moline, Ill.) under a pressure of 350-400 psi for 20 minutes and the pressure was quickly released to disrupt cells. To completely disrupt cells, the nitrogen cavitation was repeated twice. The resulting suspension was added with 2.5 mM EDTA and was centrifuged at 2,000 g at 4° C. for 30 minutes. The resulting supernatant was centrifuged at 100,000 g at 4° C. for 30 minutes (Ultra TLX Beckman). After the supernatant was removed, a pellet was suspended in 2 ml of HBSS. The suspension at 37° C. was quickly mixed with 10 μl at 1 mM dye (DiO or Dil) solution or 30 μl at 5 mg/ml of TPCA-1 and the suspension was then incubated at 37° C. for 30 minutes in a water-bath. To remove free dye molecules, the suspension was centrifuged at 100,000 g twice and the pellet was suspended in HBSS. The suspension was extruded through a membrane with 0.2 μm pores using an extruder (Avanti polar lipids, Inc., Alabaster, Ala.) to make uniform size of vesicles. Erythrocyte vesicles were similarly prepared.

Quantification of proteins and DNA of membrane formed vesicles: After the vesicles were made, the protein concentration was determined by BCA assay. Double strain DNA concentration was also determined by QuantiFluor® dsDNA assay.

Efficiency of generating membrane vesicles: The total cell lysis mass and the final vesicles were weighed after lyophilization, respectively. In particular, 66 mg of total cell lysis and 1 mg of cell membrane-formed vesicles were observed after subtracting the buffer, so the vesicles had 1.5% of total cell mass. As plasma membrane makes up approximately 2-3% of the cell mass, it was concluded that 50-75% of cell membrane was used to generate the vesicles using nitrogen cavitation to make vesicle compositions. After quantifying proteins of the vesicles using BCA assay, 0.5 mg of proteins were obtained in the vesicles, equating to about 50% of proteins in vesicles.

Vesicle size and Zeta potential: The vesicles were characterized using dynamic light scattering and TEM. A drop of the vesicle solution was deposited on a carbon-coated grid. Approximately 5 minutes after the sample was deposited, the grid was rinsed and fixed by formaldehyde solution (4%). A drop of 1% uranyl acetate to stain was added to the grid. The grid was subsequently dried and visualized using FEI Technai G2 20 Twin. The particle size and Zeta potential were also measured by Malvern Zetasizer Nano ZS90 (Westborough, Mass.).

Endothelial cell culture and their activation with TNF-α challenge: Human umbilical vein endothelial cells (HUVECs) were cultured in the EBM medium supplemented with a kit including FBS, rhEGF, hydrocortisone, GA-100, bovine brain extract and ascorbic acid. To activate the expression of ICAM-1, 100 ng/ml TNF-α was added into the medium and at defined time points the ICAM-1 expression level was determined by Western blot.

Vesicle uptake by HUVECs: HUVECs at a concentration of 1.5×10⁵/well were seeded on a coverslip in a 12-well plate and 60 μl of vesicles at 2 mg/ml were added into each well and incubated for 50 min. Cells were washed twice with PBS and fixed with 4% PFA for 10 min on ice. After washed twice with PBS, the cells were mounted on a slide with a mounting reagent containing DAPI (Life Technologies, Grand Island, N.Y.) and imaged using a confocal microscope (Olympus Fluorview FV1000). To quantitatively analyze the vesicle uptake by HUVECs, an equal fluorescence intensity of HL 60 vesicles and erythrocyte vesicles was used to incubate with HUVECs, 4 hours after treatment with 100 ng/ml of TNF-α. The cells were collected and analyzed by a flow cytometer (Accuri 6, BD, USA). The mean fluorescence intensity was measured to represent the uptake of vesicles in cells after subtracting the background.

Determination of drug loading in vesicles: The TPCA-1-loaded vesicles were treated with methanol to extract TPCA-1, and the concentration of TPCA-1 was measured using acquity of ultra-performance liquid chromatography system (Waters, Milford, Mass.). The drug was separated using a BEH C18 column (50 mm×2.1 mm) and detected at a wave length of 310 nm. The peak of TPCA-1 was confirmed by a G2-S Q mass spectrometer. The flow phase was prepared with 30% methanol in water and the flow speed was set at 0.5 ml/min.

ICAM-1 expression on HUVECs after treatment by TPCA-1 loaded vesicles: After 3 hours of treatment with TNF-α (100 ng/ml), HUVECs were incubated with 450 ng/ml of TPCA-1 loaded in HL60 cell- or erythrocyte-membrane vesicles, in the presence of TNF-α (100 ng/ml). Approximately 4 hours later, the cells were harvested and lysated to analyze ICAM-1 expression using Western Blot.

Mice: Adult CD1 mice (25-32 g) were purchased from Harlan Labs (Madison, Wis.). The mice were maintained in polyethylene cages with stainless steel lids at 20° C. with a 12 hour light/dark cycle and covered with a filter cap. Animals were fed with food and water ad lib. The Washington State University Institutional Animal Care and Use Committee approved all animal care and experimental protocols used in the studies. All experiments were made under anesthesia using intraperitoneal injection of the mixture of ketamine (120 mg/kg) and xylazine (6 mg/kg) in saline.

Intravital Microscopy of Live Mouse Cremaster Venules: Using intravital microscopy, it was real-time visualized how the membrane-formed vesicles interacted with inflamed vasculature in a live mouse. TNF-α (500 ng in 250 μl saline) was intrascrotally injected into a mouse to cause vascular inflammation where endothelium highly expressed ICAM-1. At 3 hours after TNF-α injection, the mouse was anesthetized with intraperitoneal injection of the mixture of ketamine (120 mg/kg) and xylazine (6 mg/kg), and maintained at 37° C. on a thermo-controlled rodent blanket. A tracheal tube was inserted and a right jugular vein was cannulated for infusion of vesicles, or antibodies. After the scrotum was incised, the testicle and surrounding cremaster muscles were exteriorized onto an intravital microscopy tray. The cremaster preparation was superfused with thermo-controlled (37° C.) and aerated (95% N₂, 5% CO₂) bicarbonate-buffered saline throughout the experiment. Images were recorded using a Nikon A1R⁺ laser scanning confocal microscope with a resonant scanner.

To study the adherence of vesicles to an inflamed venule whose size is from 20-30 μm, Alex Fluor-647 anti-mouse CD31 antibody (2.5ug per mouse) and HL 60 vesicles labeled with DiO (0.1 mg/mouse) were simultaneously infused into the TNF-α-treated mouse, water immersion objective with NA=1.1 was used to image cremaster venules. Two lasers at 640nm and 488 nm simultaneously excited cremaster tissues to image venules and vesicles at 10 frames/second for 512×512 pixels. Images were analyzed using Nikon software. To compare the adherence of HL 60 membrane vesicles to inflamed venules with erythrocyte vesicles, HL 60 vesicles labeled with DiL (560 nm) and erythrocyte vesicles labeled with DiO (488 nm) were simultaneously infused at the same concentration in a mouse. The intravital images were quantified using Nikon software (NIS Elements) to measure fluorescence intensity of vesicles. To address whether vesicles interact with resting endothelium, HL 60 and erythrocyte vesicles were injected via tail vein to a mouse without TNF-α treatment. About 1 hour after injection, cremaster tissue was exposed under an intravital microscope.

Mouse acute lung inflammatory (ALI) model: CD1 male mice were utilized. ALI was induced by intracheal spray of LPS (10 mg/kg) using a Model ICA-IC-M MicroSprayer Aerosolizer (Pen-Century). Approximately 3 hours after LPS was challenged, mice were intravenously injected with HBSS, free drug (TPCA-1), TPCA-1 loaded erythrocyte vesicles and TPCA-1 loaded HL 60 vesicles at two doses of 0.33 and 1 mg/ kg.

Bronchoalveolar lavage fluid collection and cell count: At 13 hours post-LPS administration, mice were anesthetized with an i.p. injection of ketamine and xylazine mixture. The trachea was cannulated, and 1 ml HBSS was infused intratracheally and withdrawn to obtain lavage fluid. This procedure was repeated twice. The bronchoalveolar lavage (BAL) fluid was centrifuged at 420 g for 4 min, and cell pellets were suspended in 0.7 ml red blood cell lysis buffer (Qiagen, Valencia, Calif.). After 30 minutes, the cells were pelleted by centrifugation at 420 g for 4 minutes, and suspended in 0.5 ml HBSS. The total cell number was determined with a hemocytometer. Cell suspensions were diluted to a final concentration at 1×10⁵cells/ml and a 200-μl of the suspension was spun onto a slide at 700 rpm for 5 minutes using a cytocentrifuge (Shandon, Southern Sewickley, Pa.). The slides were stained with Diff-Quick dye, and examined at a magnification of 400 by light microscope. The percentages of neutrophils were determined after counting 200 cells in randomly selected fields.

Cytokine generation: Cytokine levels in the BAL were determined using commercial ELISA kits for TNF-α and IL-6 (Biolegend, San Diego, Calif.) according to the manufacturer's instructions. The triplicate experiment was conducted.

Measurement of lung vascular permeability: The permeability was evaluated via the larvage protein content change. The total protein concentrations were determined using a BCA protein assay kit (Thermo scientific, Rockford, Ill.).

Statistical Analysis: Data are expressed as mean±SD. Statistical analysis was conducted using one or two-way T-test using Origin 8.5. p values<0.05 are considered significant.

### EXAMPLE 2

### Model for Formation of Vesicle Compositions

In the instant example, a novel platform for exploiting a diseased cell as a building block to create cell membrane-formed nanosized vesicles possessing intact targeting molecules is demonstrated. Using HL 60 cells, similar to human neutrophils, as a model, membrane-formed vesicles were generated with a size of 200 nm in diameter, and 50-75% of cell plasma membrane was used to make vesicles. The approach provides a very efficient approach compared with other methods, such as chemical agents to disrupt cells.

Intravital microscopy of cremaster venules of a live mouse shows the ability of these vesicles that selectively bind inflamed vasculature. In in vitro experiments of HUVECs treated TNF-α, HL-60 vesicles loaded with TPCA-1 dramatically reduced ICAM-1 expression. When infusing these vesicles into a mouse challenged by LPS, the vesicles can markedly mitigate acute lung inflammation and injury.

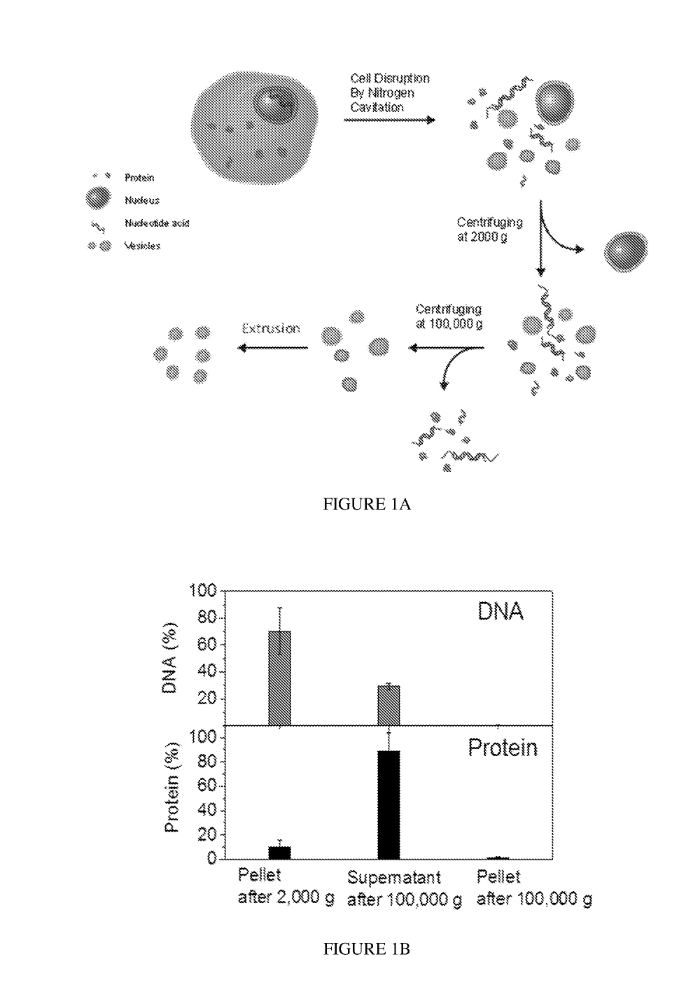

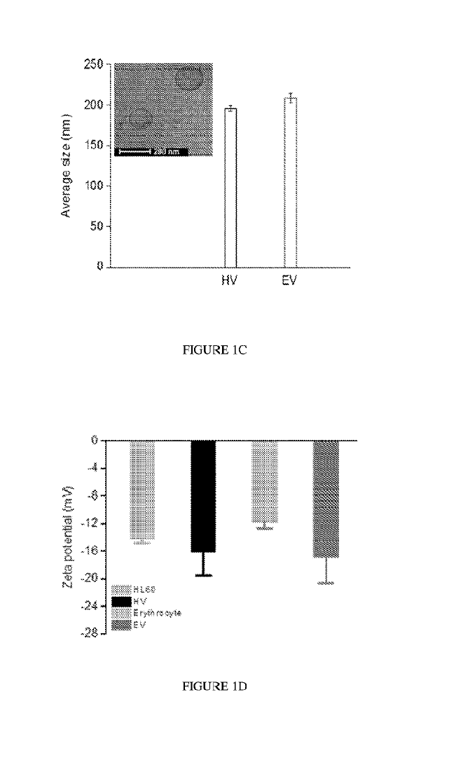

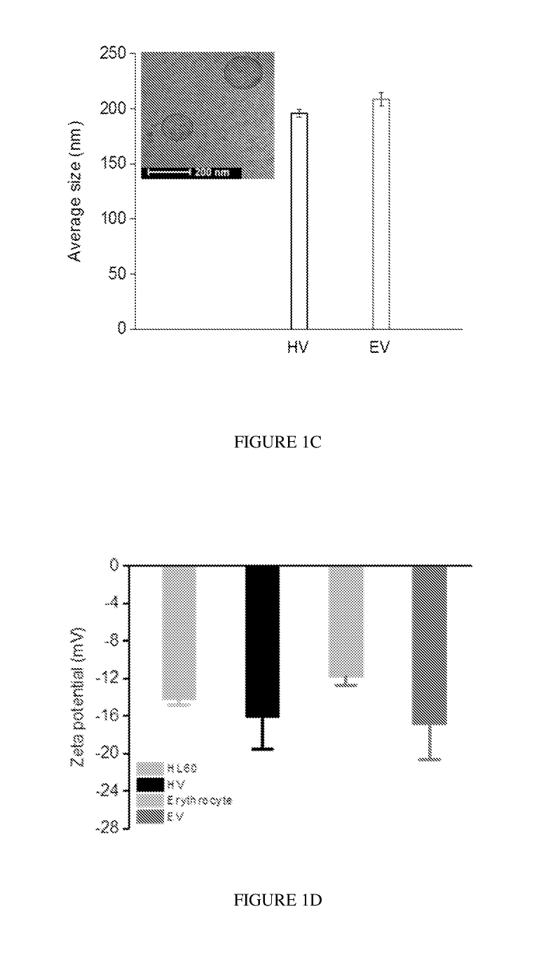

A general method was developed to create cell membrane-formed vesicles using a mechanical force generated by nitrogen cavitation which can disrupt cells and maintains intact biological functions of membrane molecules (see FIG. 1A). After a cell was disrupted by nitrogen cavitation under a high pressure of 350 psi, the resulting solution contained membrane vesicles, intracellular molecules and nucleus. A differential centrifugation approach was used to obtain the needed vesicles. The products after each step of centrifugation were determined by using protein and DNA assays (see FIG. 1B). A pellet after 2,000 g showed a major content of DNA which contained 70% of cell nuclear molecules, but only 10% of proteins. The supernatant was further centrifuged at 100,000 g. The resulting supernatant showed 88% of proteins and 30% of DNA in a whole cell lysis. In contrast, the pellet after 100,000 g centrifugation unlikely contained DNA molecules, and 1.3% of proteins of a whole cell lysis existed in the pellet which could be cell membrane-formed vesicles. The pellet was weighed after lyophilization and the proteins were quantified using BCA assay. It is found that the proteins were 50% of total mass of the pellet (Methods in Supplementary), which is consistent with the estimate that a cell plasma membrane is comprised of approximately 50% lipid and 50% protein by weight. The result suggests that the final product is mainly composed of plasma membrane, and is also consistent with human neutrophil membrane vesicles generated by nitrogen cavitation in the application of membrane protein isolation.

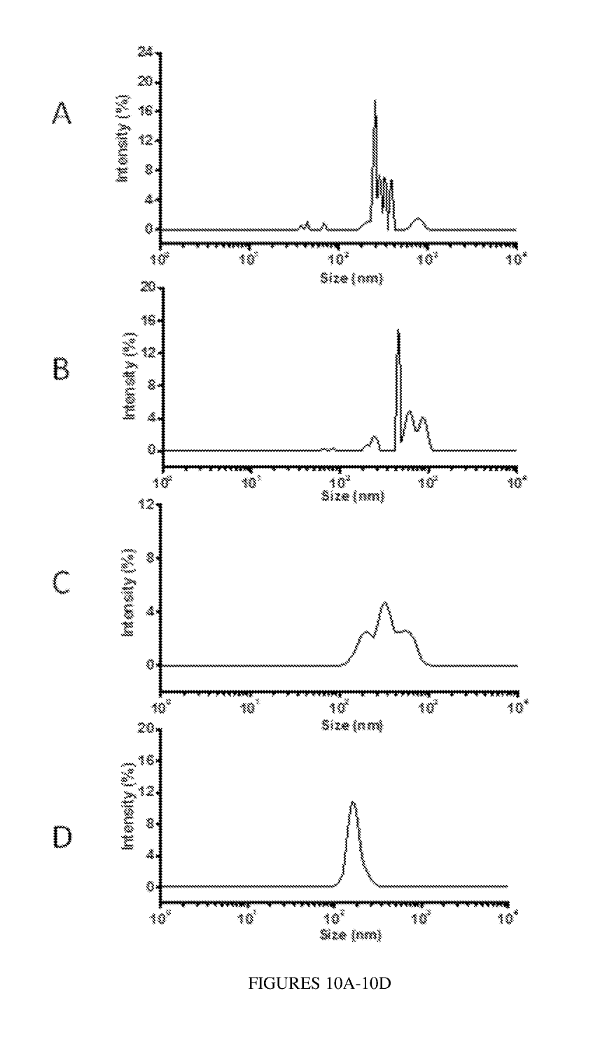

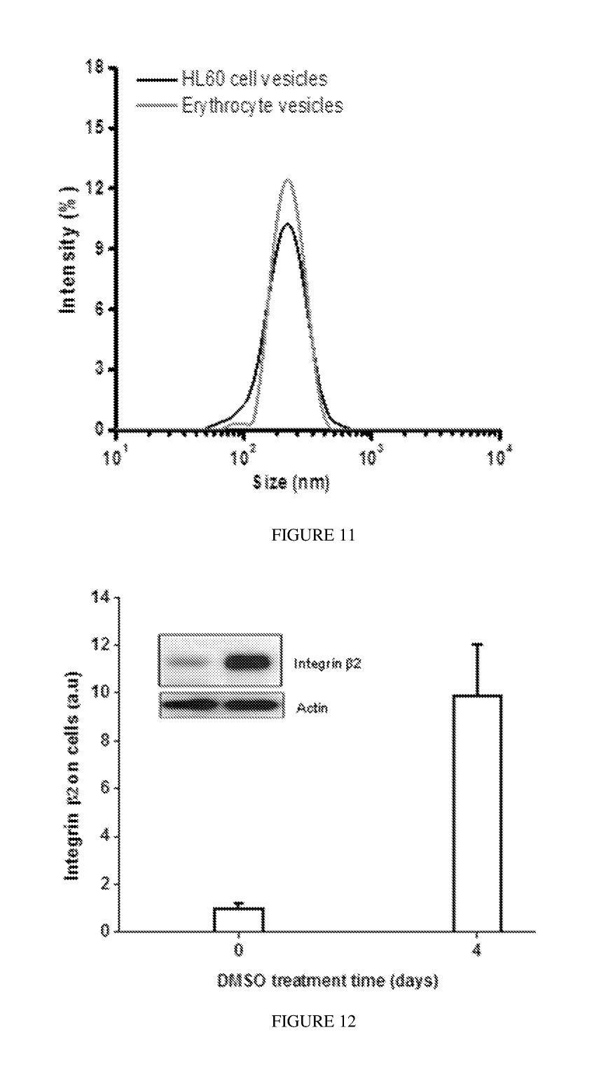

It was also determined that 50-75% of HL 60 cell plasma membrane was successfully used to form vesicles, so the instant approach is highly efficient to produce membrane-formed vesicles. Using dynamic light scattering we measured the sizes of the product of each step after centrifugation (see FIG. 10), showing a broad range of particle size. After extruded through a membrane with pores of 200 nm, the vesicles gave rise to a uniform size (see FIG. 10). In this study, HL 60 cells were used as a model because their functions are similar to neutrophils in vivo²², and chose erythrocytes (red blood cells) as a negative control. The dynamic light scattering and TEM images (see FIG. 1C and FIG. 11) showed a shell structure of vesicles with a size of 200 nm in diameter. The zeta potential of HL 60 cell membrane-formed vesicles (HVs) (−16 mV) was close to their parent cells (−14 mV) (see FIG. 1D), and the similar result appeared for erythrocyte membrane-formed vesicles (EVs) (see FIGS. 1C and 1D). Thus, the generated vesicles originated from their source cell membranes.

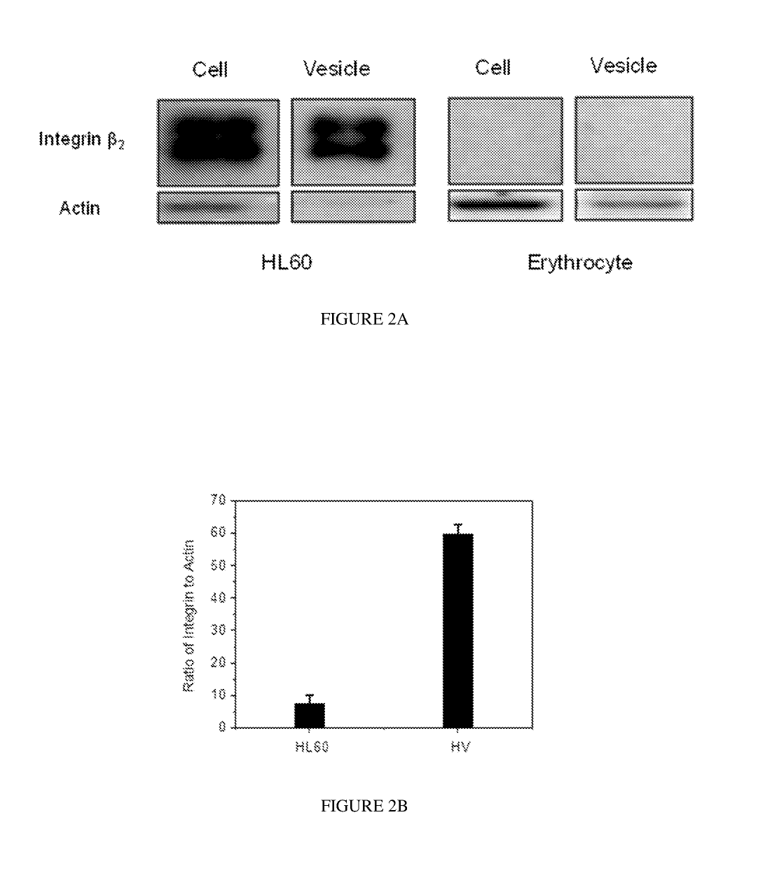

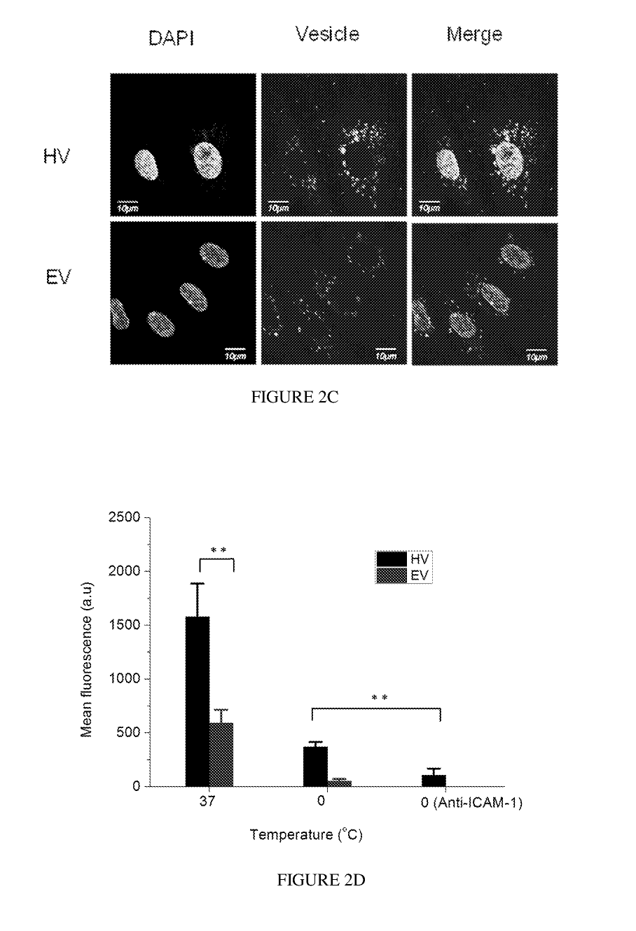

DMSO (dimethyl sulfoxide) treatment can activate HL 60 cells to dramatically express integrin β₂ (see FIG. 12) which can be used to study neutrophil functions, such as adherence to endothelium. HL 60 vesicles highly expressed integrin β₂ and showed a large ratio of integrin β₂ to actin (intracellular proteins) compared to their source cells (see FIG. 2A and FIG. 2B), suggesting the vesicles might possess a higher density of integrin after the formation of vesicles. In contrast, erythrocytes and their vesicles did not express integrin β₂, so the erythrocyte vesicles (EV) are an excellent control to address the ability of HL 60 vesicles that can selectively target activated endothelium.

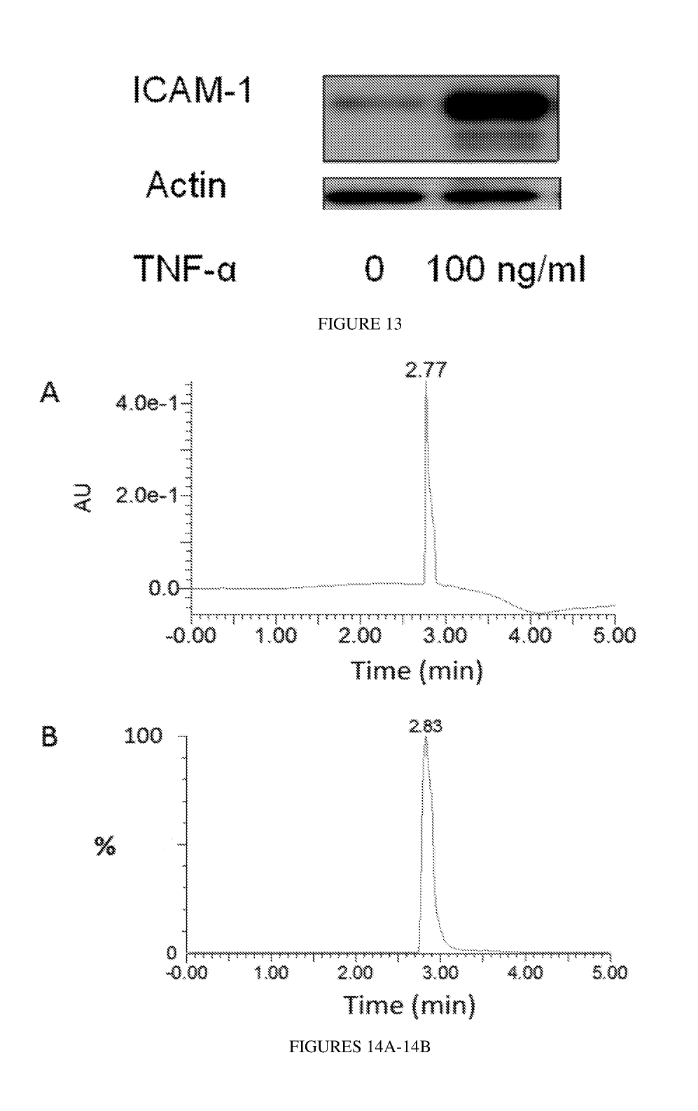

To study whether HL 60 vesicles can bind to activated endothelium, vesicles were fluorescently labeled with a lipid dye. HUEVCs were treated with TNF-α (100 ng/ml) to activate endothelium to express ICAM-1 (see FIG. 13), and then HUVECs were incubated with HL 60 vesicles or erythrocyte vesicles for 50 minutes. The confocal images (see FIG. 2C) of inside cells showed that HL 60 vesicles were more efficiently internalized by HUVECs than erythrocyte vesicles. To quantitative analysis of vesicle uptake by HUVECs, the flow cytometry was used and measured the mean fluorescence intensity of vesicles per cell (see FIG. 2D). The uptake of HL 60 vesicles in HUVECs was three-fold more than erythrocyte vesicles, indicating that the binding of integrin β₂ to ICAM-1 is required for vesicle internalization.

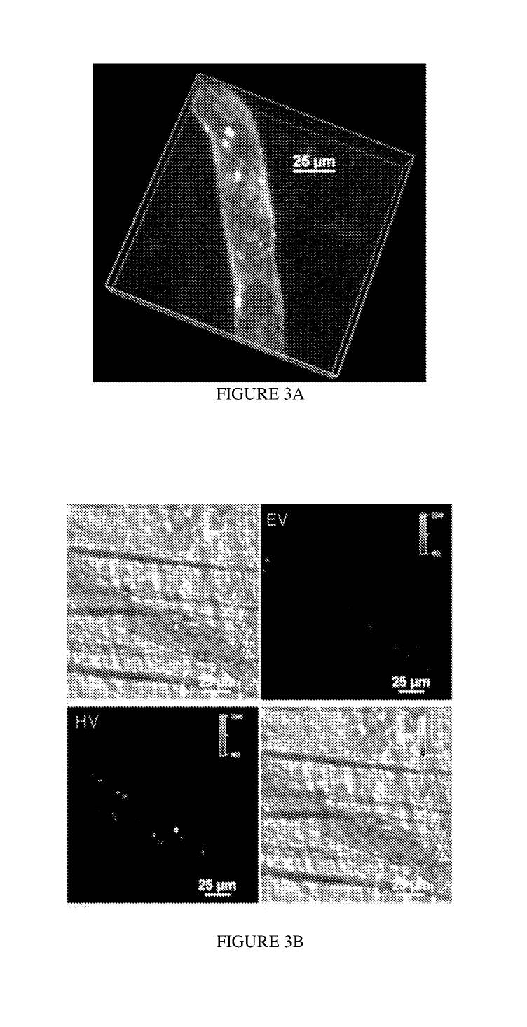

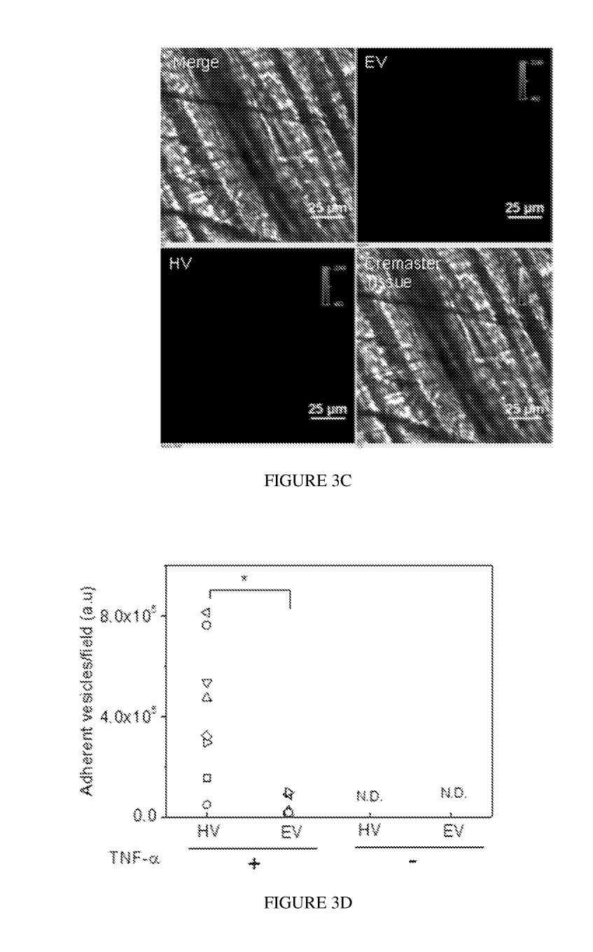

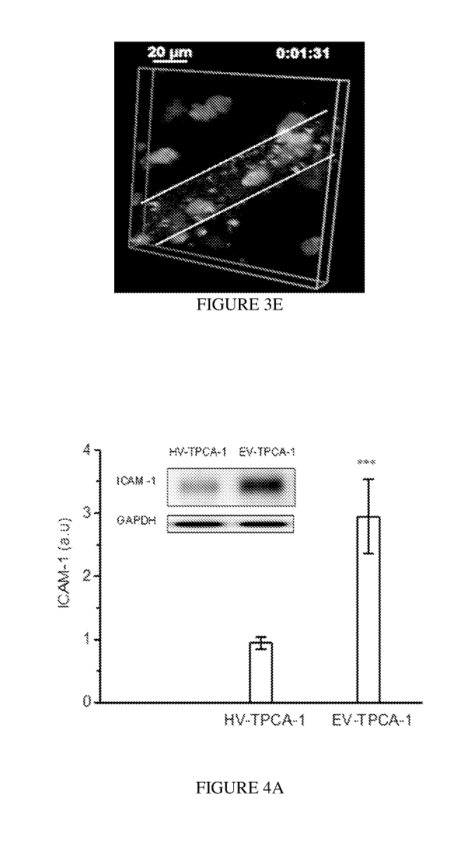

Using intravital confocal microscopy of live mouse cremaster venules, it was determined whether HL 60 vesicles interacted with inflamed vessels in vivo. Approximately 3 hours after intrascrotal injection of TNF-α, which causes the up-regulation of ICAM-1 on endothelium vessels, intravenously infused HL 60 vesicles were adherent to cremaster venules fluorescently labeled by anti-CD31 (PECAM-1), a marker of endothelium (see FIG. 3A). To define whether integrin β₂ is required for the adherence of HL 60 vesicles to activated endothelium, HL 60 vesicles and erythrocyte vesicles were simultaneously infused into a mouse intrascrotally treated with TNF-α. Many puncta of HL 60 vesicles were observed adherent to venules compared to erythrocyte vesicles (see FIG. 3B). However, in a mouse without TNF-α treatment, neither HL 60 vesicles nor erythrocyte vesicles were observed to be adherent to the vessel wall (see FIG. 3C). After normalizing fluorescent intensity of HL 60 and erythrocyte vesicles, adherent vesicles per image field of intravital microscopy were quantified (see FIG. 3D). Integrin β₂ was suggested to be required for the binding of HL60 cell vesicles to activated endothelium. An endothelial vessel is so thin that it is difficult to visualize the vesicle uptake in the endothelial vessel using intravital microscopy. However, in a 2-3 hour period of observation, adherent vesicles rarely detached from vessel walls, implying that the adherent vesicles were likely to be internalized by endothelium. Some puncta were also observed that were larger than the vesicle physical size (see FIG. 3A and FIG. 3B). This could be related to the infusion of HL 60 vesicles with large organelles in a cell after vesicle internalization. Another possibility may be relevant to multiple vesicles closely adherent to endothelium due to heterogeneous distribution of adherent molecules expressed in vivo.

At a site of inflammation, neutrophils are adherent to vessel walls, so it is not clear whether HL 60 cell vesicles interact with neutrophils. Alex-fluor-488-labeled anti-Gr-1 antibody to mark neutrophils, and fluorescently-labeled HL 60 vesicles were simultaneously infused in a mouse. The HL 60 vesicles did not interact with neutrophils, but accumulated in an inflamed location close to neutrophils (see FIG. 3E), showing that HL 60 vesicles are capable of targeting the inflamed vasculature.

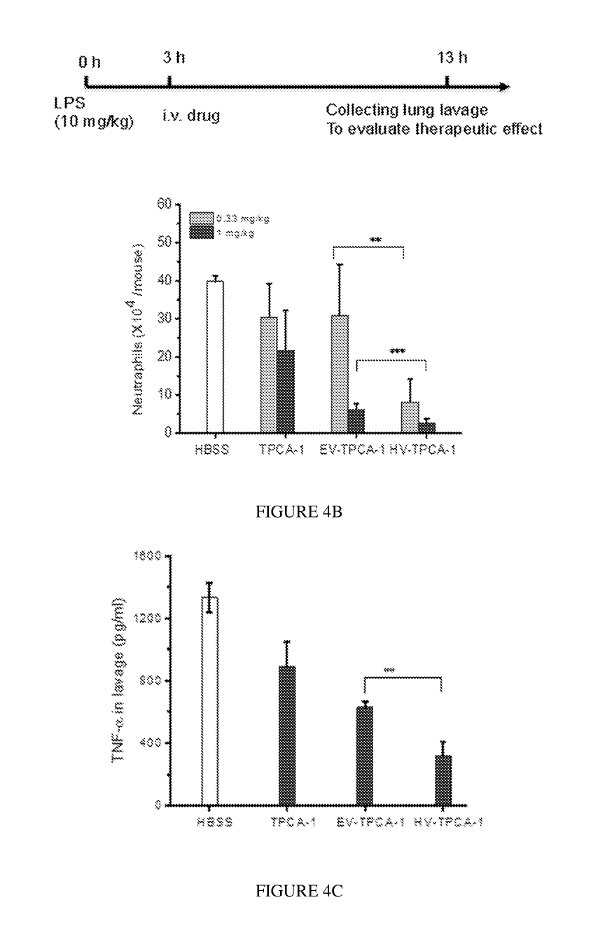

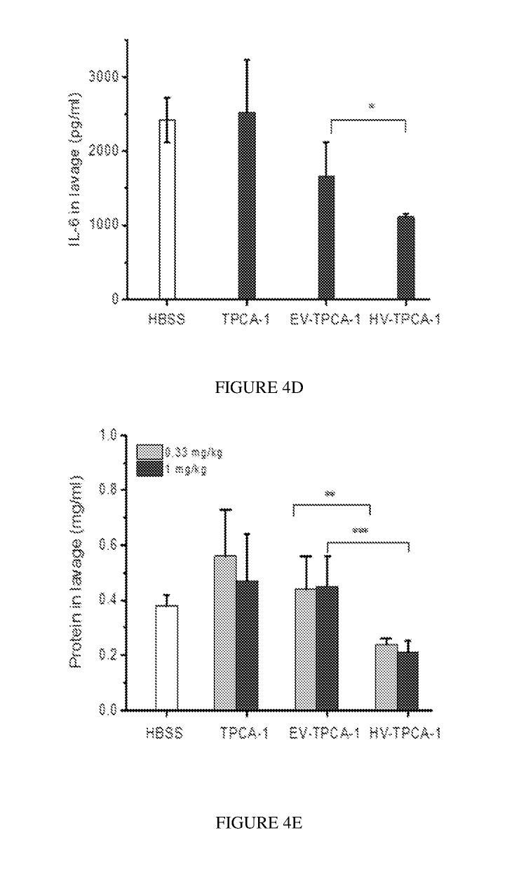

Next, it was examined whether HL 60 vesicles as a carrier were able to deliver therapeutic to inflamed vasculature to attenuate inflammation. To this end, the vesicles were loaded with TPCA-1 (2-[(Aminocarbonyl)amino]-5-(4-fluorophyneyl)-3-thiophenecarboxamide), which is a NF-κB inhibitor (see FIG. 14). The NF-κB pathway is a central regulator to control inflammation response when infection or tissue damage occurs. After HL 60 or erythrocyte vesicles loaded with TPCA-1 were incubated with TNF-α-activated HUVECs, HL 60 vesicles dramatically reduced ICAM-1 expression on endothelial cells (see FIG. 4A).

Acute lung inflammation and injury, and its most severe form, acute respiratory distress syndrome (ARDS), cause 40% mortality in approximately 200,000 patient annually in the United States. The pathological underlying is primarily linked to cytokine storms produced by lung residing macrophages leading to activation of endothelium that recruits neutrophils into a lung. The NF-κB pathway is a central regulator to activate endothelium to express ICAM-1 for neutrophil recruitment, so it was examined whether HL 60 vesicles loaded with TPCA-1 could alleviate vascular inflammation. About 3 hours after instillation of LPS into a mouse lung, either HL 60 vesicles (HV), erythrocyte vesicles (EV), or free drug was administered. The therapeutic effects at 13 hours after LPS injection were investigated. Infiltration of neutrophils in a lung dramatically reduced after HL 60 vesicle administration compared to erythrocyte vesicles or free drug, and was also showed a dose-dependent manner (see FIG. 4B). Unexpectedly, even at 0.33 mg/kg, HL 60 vesicles showed marked therapy but erythrocyte vesicles and free drug rarely showed this benefit. The inflammatory factors such as TNF-α and IL-6 showed that the lung inflammation markedly attenuated after treatment of HL 60 vesicles compared with erythrocyte vesicles or free drug (see FIGS. 4C and 4D). In addition, TPCA-1 loaded HL 60 vesicles lowered the lung permeability compared with free drug or erythrocyte vesicles (see FIG. 4E), representing that HL 60 vesicles can improve the lung integrity, thus preventing lung injury from edema.

### EXAMPLE 3

### Loading of Agents in Vesicle Compositions

TPCA-1 drugs are shown to be entrapped in the membrane of vesicles. It was hypothesized that pH values could drive the drug loading efficiency. A pH value of cell-membrane-formed vesicles was increased to 9.5 using sonication or the buffer used in formation of cell vesicles. The pH value inside of vesicles was confirmed using a pH sensor. In controls, the vesicles had a pH of 7. Piceatannol was loaded at 1 mg/ml, and lowered the pH at 4. About 2 hours after incubation, the drugs in the vesicles were quantified. The result showed that when the pH value was increased, the drug loading efficiency was increased by 3-4 fold. In the instant example, the pH gradient drives the drug loading.

Furthermore, the loading efficiency of acid or base drugs can be successfully increased.

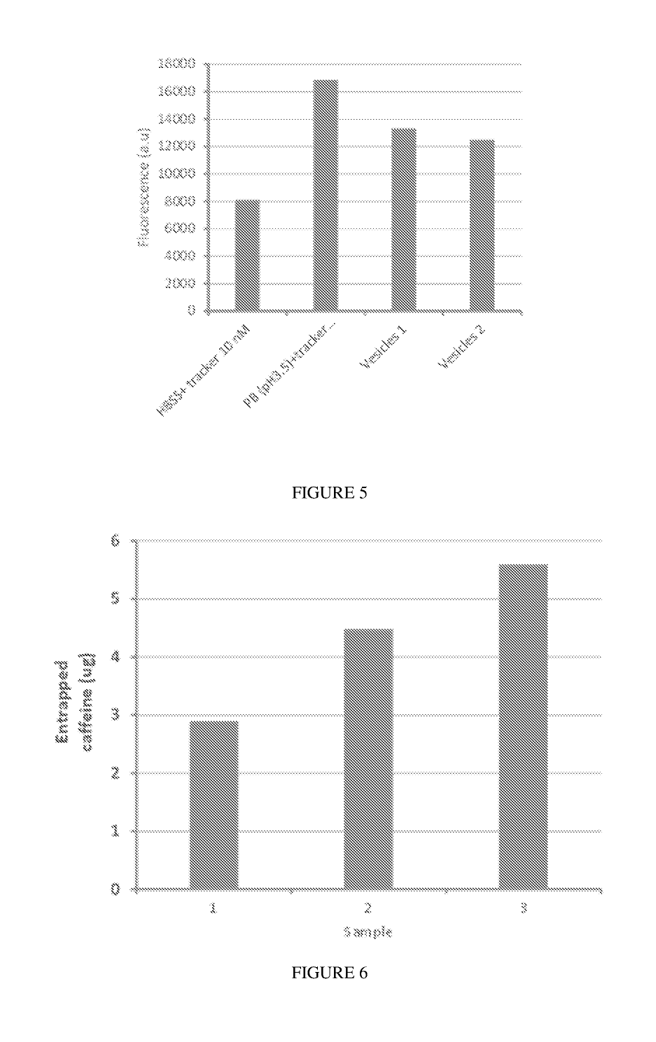

Loading basic drugs inside of vesicles: Using a sonication approach, pH values inside of vesicles can be modified. After HL 60 cell-membrane-formed vesicles were obtained, they were suspended in sodium phosphate buffer (20 mM, pH=3.5) and sonicated at the different power. The vesicles were obtained by centrifugation and resuspended in HBSS buffer (pH=7.5) with pH sensors, and measured fluorescence intensity at 511 nm to show the pH changes. FIG. 5 shows that when the pH values become acidic, the fluorescence increases. It was also observed that using the sonication approach, the pH values inside of vesicles can be changed. Thereafter it was examined whether a base drug, such as caffeine, can be loaded inside of vesicles. Vesicles of pH=3.5 were obtained and incubated with caffeine at the same concentration, and the drug solution pH value was changed to make a gradient. FIG. 6 shows that when caffeine solution was changed at pH=9, or sonicated vesicles at caffeine solution in HBSS of pH=7.5, the drug loading can be dramatically increased compared with the control of in caffeine HBSS at pH=7.5

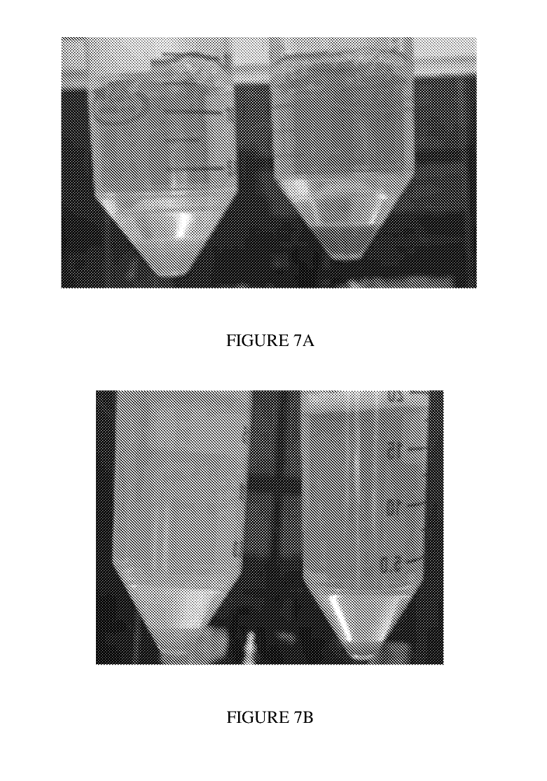

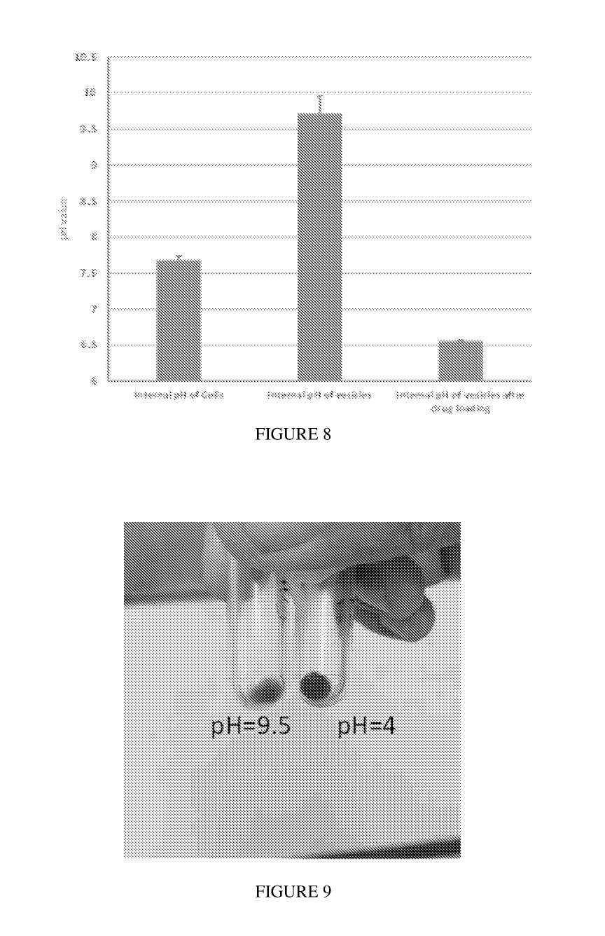

Loading acid drugs inside of vesicles: In this approach, HL 60 cells were suspended in the base buffer (pH=9.5). When cells were disrupted, the formed vesicles should have same the pH value as the used buffer. To prove this concept, a pH sensor, SNARF-1AM, was used to measure the pH values inside of vesicles. FIG. 7A shows after SNARF-1AM was loaded in HL 60 cells, and centrifuged cells down, most SNARF-1 appeared in the cells. After disrupting HL 60 cells and centrifuging to obtain the vesicles, the sensors were observed to exist in the vesicles (see FIG. 7B). FIG. 8 demonstrates that the vesicles have the same of pH value as the used buffer (pH=9.5). Furthermore, piceatannol (anti-inflammation drug) can successfully be loaded in these vesicles as the pH value inside of vesicles was shown to decrease because piceatannol neutralized the buffer. In controls, the vesicles kept the pH value at 9.5, but piceatannol solution with a pH at 9.5 was kept. Compared with the sample of a pH gradient where piceatannol with a pH at 4, the loading efficiency can be increased by 3-4 folds.

Another fluorescent dye (bromophenol blue, an acidic molecule) was also tested the difference of loading efficiency can be observed. In the similar experiment above, the controls kept the bromophenol blue solution at pH=9.5 when loaded into vesicles (inside vesicles with a pH=9.5). When bromophenol blue solution was decreased to a pH value of 4, the loading efficiency can be increased by 2-3 folds (see FIG. 9).

### EXAMPLE 4

### Comparison of Vesicle Compositions (HVs) With Extracellular Vesicles (EVs)

Extracellular vesicles (membrane-enclosed vesicles; “EVs”) are spontaneously released from different cell types under stimulation. EVs mediate intercellular communication from their cells of origin to target cells, and therefore could be utilized as a cargo to deliver therapeutics. However, a technical challenge is to develop approaches to efficiently and reproducibly produce EVs as safe drug carriers, because EVs contain intracellular organelles, such as endoplasmic reticulum, mitochondria, lysosomes and Golgi bodies upon production. In contrast, the instantly described method of nitrogen cavitation can efficiently produce pure extracellular vesicles because nitrogen cavitation can break cells to remove intracellular compartments, and simultaneously disrupted cell membrane forms EVs. The instant example compares the instantly described methods compared to the existing approaches.

The conventional approach to produce EVs is to culture cells and then to collect a supernatant, and finally to centrifuge to concentrate EVs. In this approach, the supernatant will contain a wide range of subcellular compartment secreted from cultured cells. To compare the production of EVs by nitrogen cavitation to the conventional approach, the instant example was performed. The cell culture medium (700 ml containing 6×10⁸ cells) of HL 60 cells was collected 4 days after DMOS treatment and centrifuged at 300 g to collect cells used for producing membrane nanovesicles using nitrogen cavitation. The supernatant was centrifuged at 2000 g to remove cell debris, and then centrifuged at 100,000 g for 1 hour to get EVs.

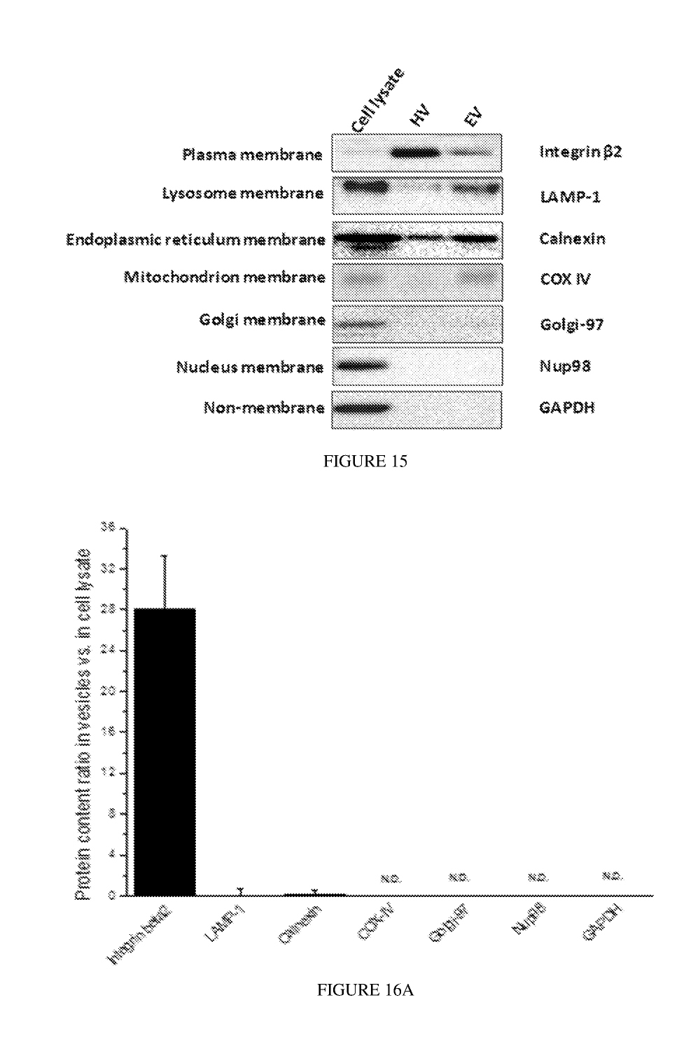

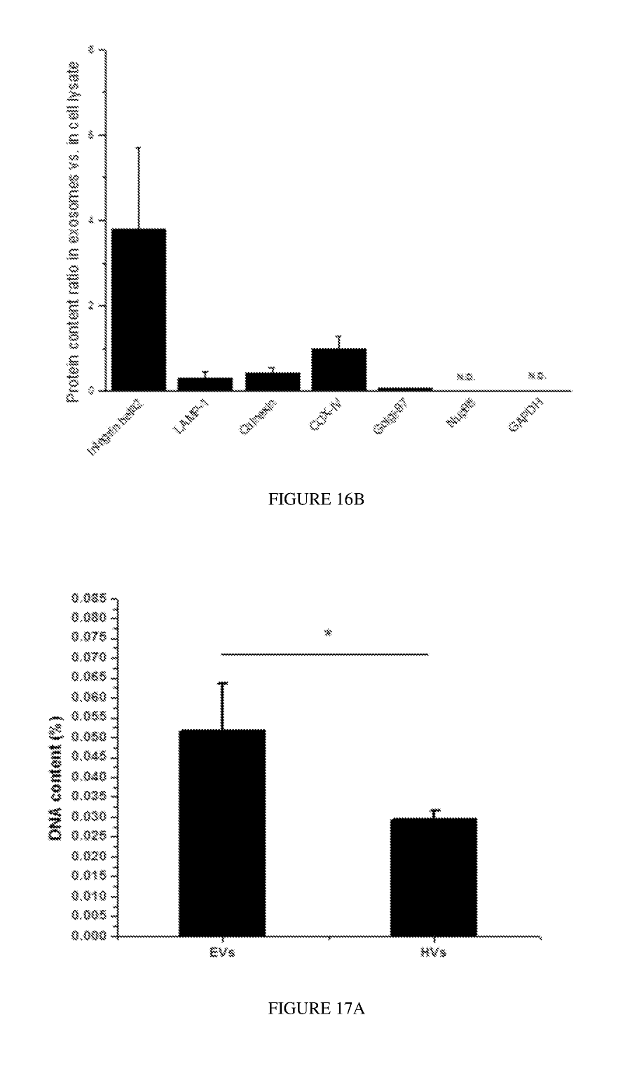

First, Western blots were performed for EVs produced by nitrogen cavitation and from the culture medium to determine the composition of membrane nanovesicles (see FIGS. 15 and 16). FIG. 15 shows the Western blot of HL 60 nanovesicles produced by nitrogen cavitation and EVs made from HL 60 culture medium. FIG. 16 shows the quantification of biomarkers observed in FIG. 7. The results demonstrate that the nitrogen cavitation approach can generate the membrane nanovesicles containing the pure plasma membrane of cells because subcellular components, such as lysosomes, endoplasmic reticulum and mitochondria, were not observed.

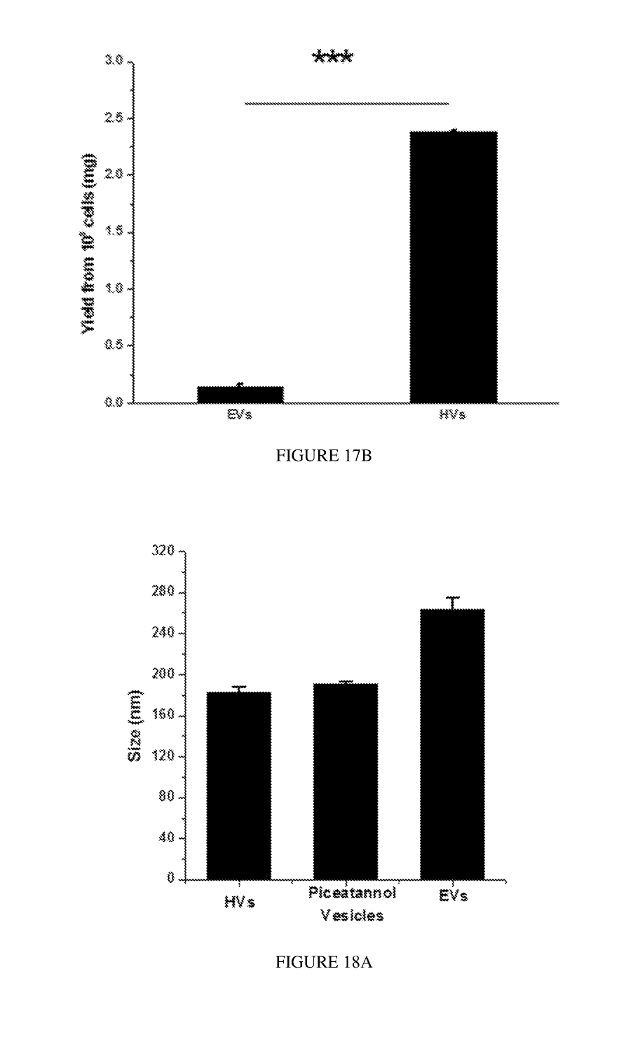

The DNA content in cell-formed nanovesicles was also evaluated because the genetic materials could cause a side effect when nanovesicles are used as delivery cargo. FIG. 17A shows that the vesicle compositions contained less DNA compared EVs made using the conventional methods. When the production of nanovesicles was investigated (FIG. 17B), the described nitrogen cavitation approach was shown to make nanovesicles more efficiently by 20 times compared with the conventional approach.

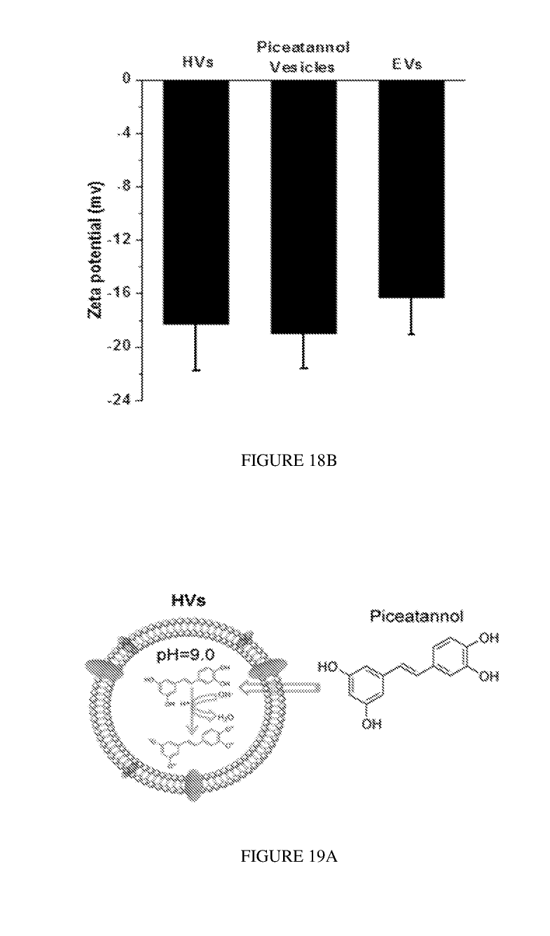

FIG. 18 shows the results of size and surface of HL 60 nanovesicles made from nitrogen cavitation and by HL60 cell culture medium. It is noted that the surface charge of nanovesicles (HVs and EVs) made by the two methods are similar, suggesting that they are made from cell plasma membrane. However, the size of the described vesicle compositions is smaller than EVs, indicating that the vesicle compositions are better for delivering therapeutics to diseased sites.

### EXAMPLE 5

### The pH Driven Loading of Agents in Vesicle Compositions

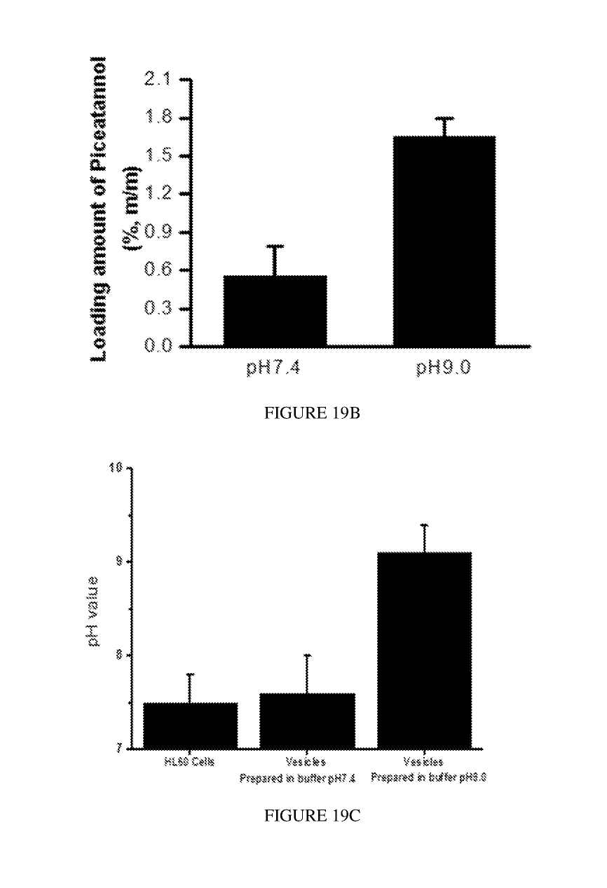

It is challenging to load therapeutics in preformed vesicle compositions. As such, means to efficiently load drugs in HL 60 vesicle compositions based on the pH gradient between intravesicles and outside of vesicles has been described. FIG. 19A shows the concept of loading the exemplary agent piceattanol in HL 60 vesicle compositions. The results (FIGS. 19B and 19C) demonstrate that piceatannol can be successfully loaded in HL 60 nanovesicles based on pH gradient.

### EXAMPLE 6

### Piceatannol-Loaded Vesicle Compositions Increase Survival in a Sepsis Model

Sepsis is a life-threatening condition that arises when the body's response to infection causes systemic organ dysfunctions and injures. The death rate could be 50-80%, such as acute lung injury, and its most severe form, acute respiratory distress syndrome (ARDS). The cause is strongly linked to immune infiltration damaging vasculature resulting in organ malfunctions.

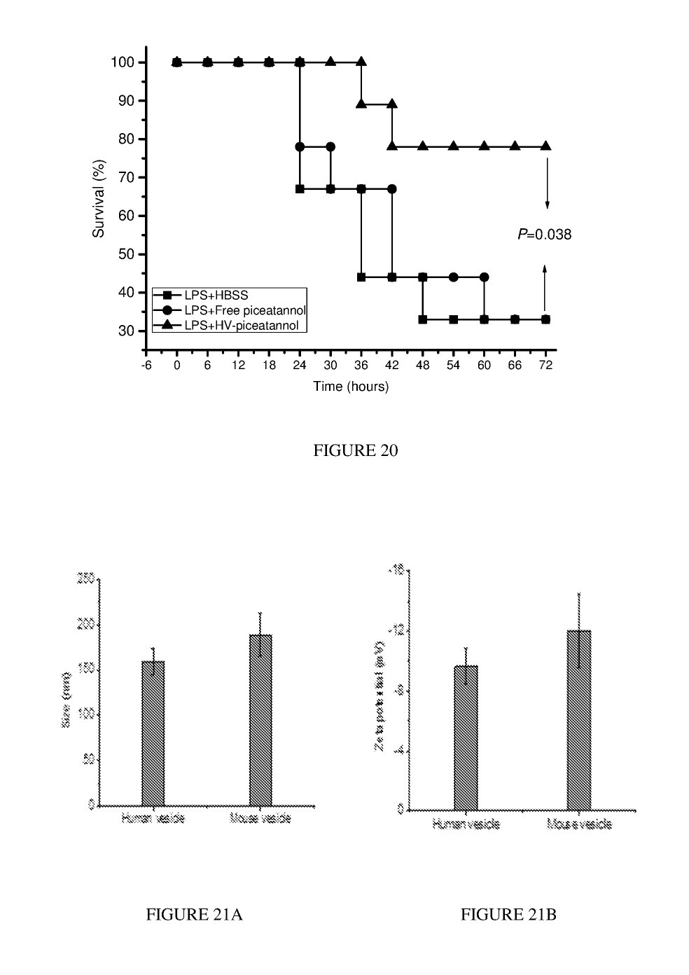

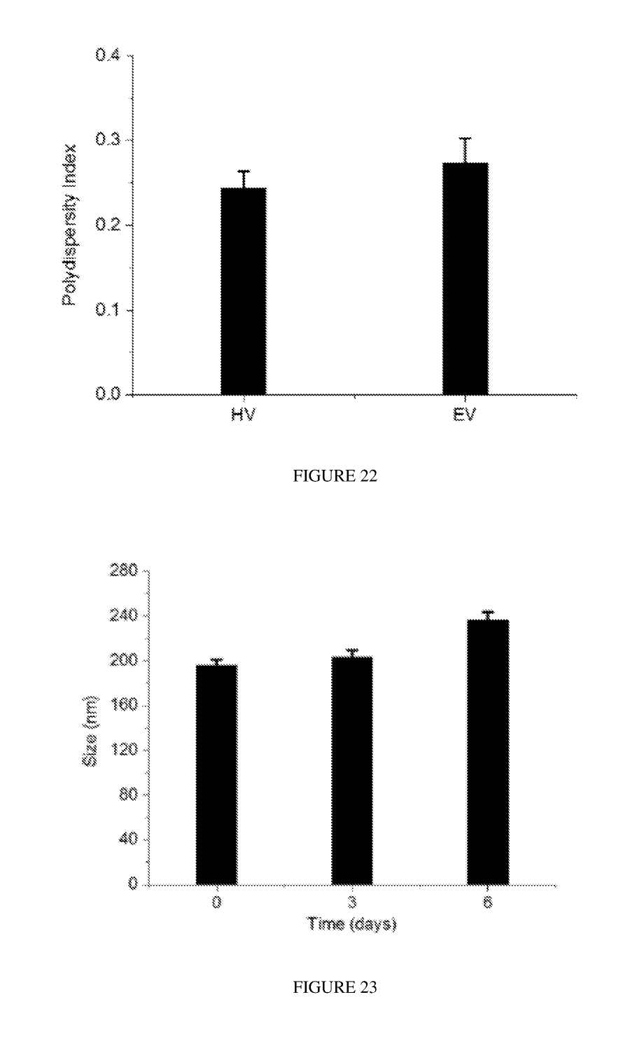

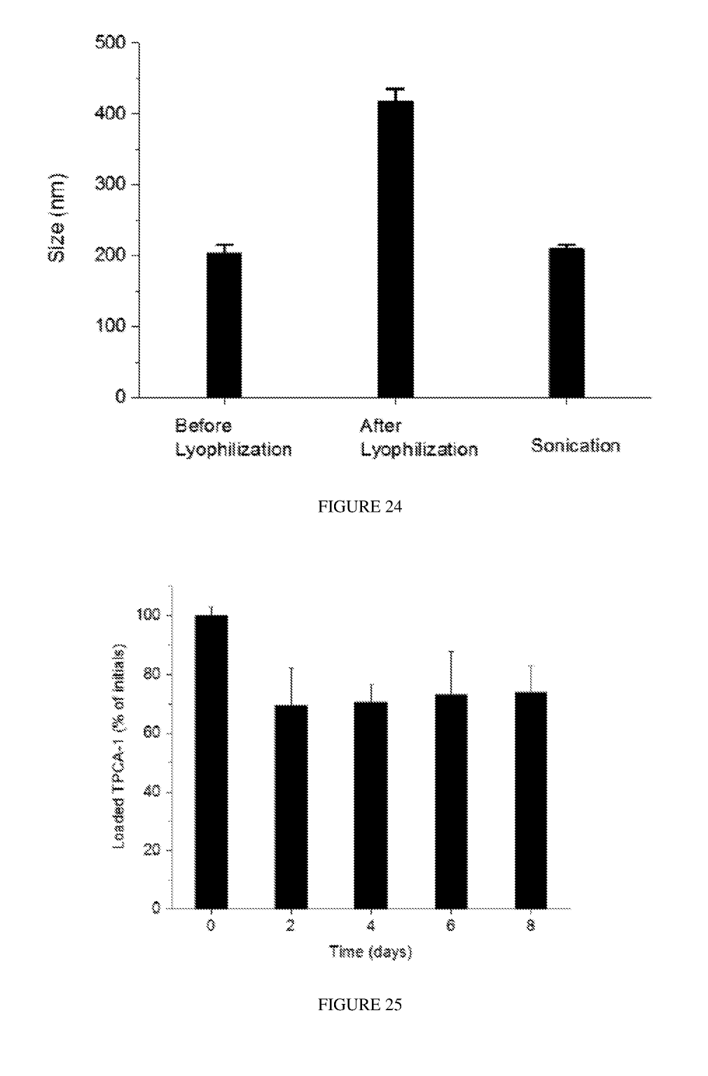

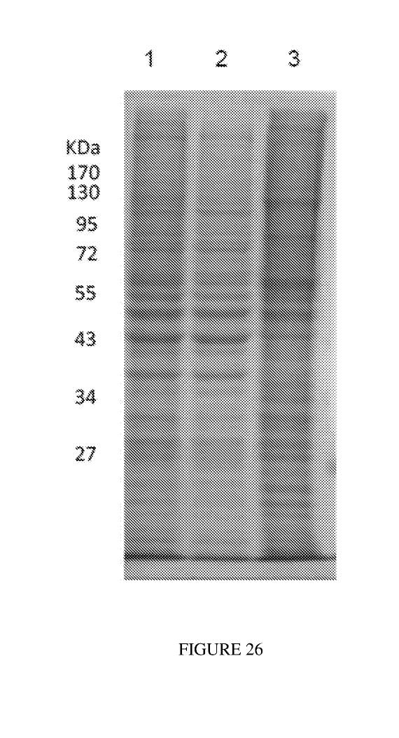

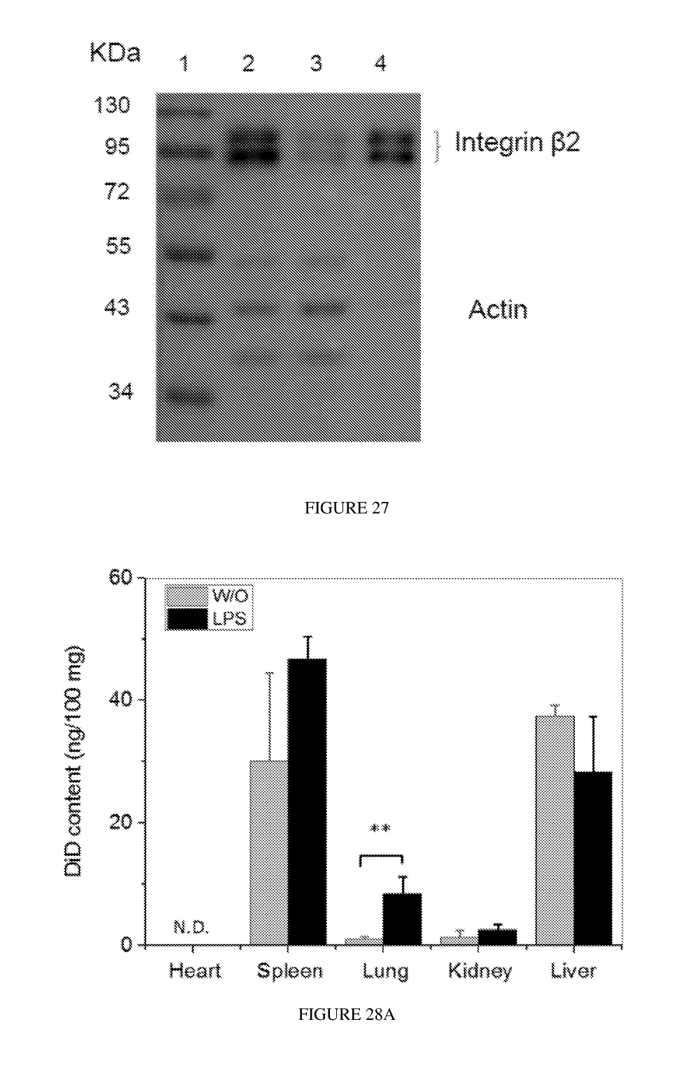

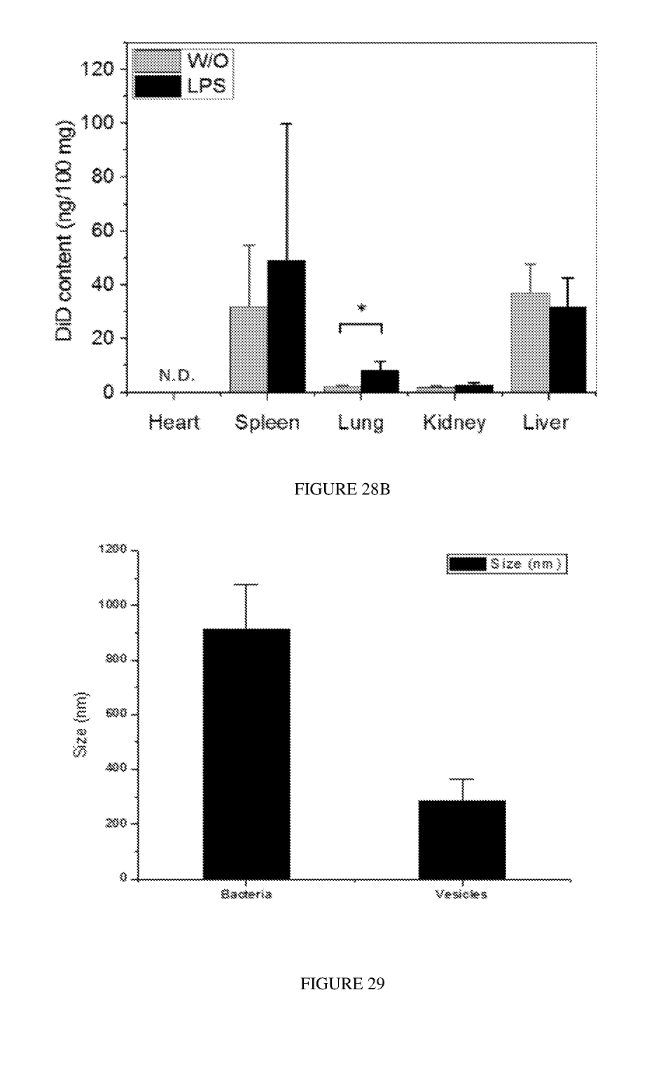

The instant example examines if the described vesicle compositions can prevent the death caused by LPS-induced sepsis. In this example, CD1 mice were randomly grouped into 3 groups (9 mice per group). Approximately 2 hours after LPS administration (22 mg/kg), three groups were intravenously received HBSS, 3 mg/kg piceatannol in HBSS, and 3 mg/kg (based on piceatannol) piceatannol-loaded vesicles, respectively. The mice were monitored for 72 hours and the survival rate was calculated. FIG. 20 shows that the piceatannol-loaded nanovesicles dramatically increased the survival of treated mice to 80% compared to free piceatannol and to controls (i.e., mice without treatment).

EXAMPLE 7

### Vesicle Compositions Made From Human Neutrophils and Mouse Neutrophils

To address the feasibility of translation of our technology, it was determined whether vesicle compositions could be formed from human or mouse primary neutrophils using nitrogen cavitation. Results demonstrated that that nanovesicles can be generated from human or mouse neutrophils using nitrogen cavitation (see FIGS. 21A and 21B).

### EXAMPLE 8

### Formation of Vesicle Compositions Comprising Bacterial Cell Membranes

Vesicle compositions of the present disclosure can be formulated by using bacterial cells. The instant example provides methods of forming vesicle compositions using the exemplary bacteria Escherichia coli and Pseudomonas aeruginosa. Methods for the instant example are similar to the nitrogen cavitation methods described in previous examples, but for the instant example Escherichia coli and Pseudomonas aeruginosa cells were utilized.

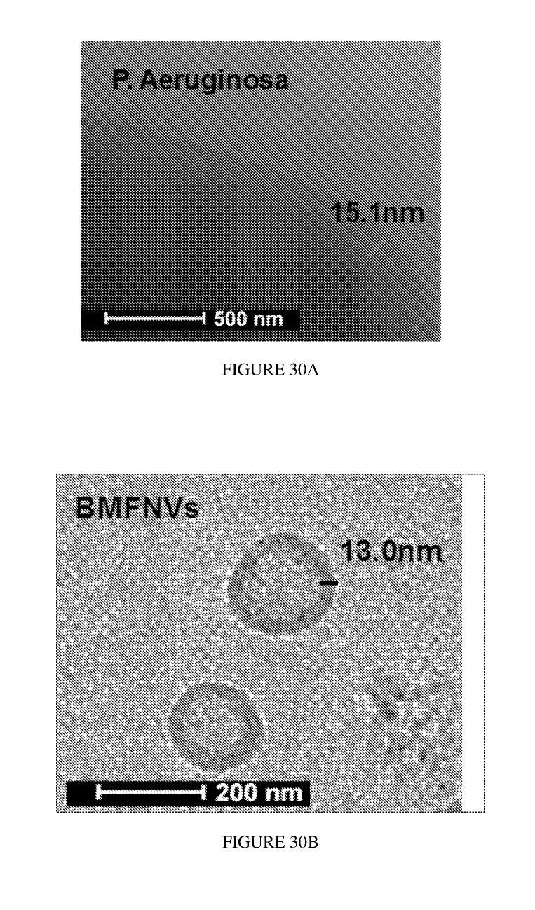

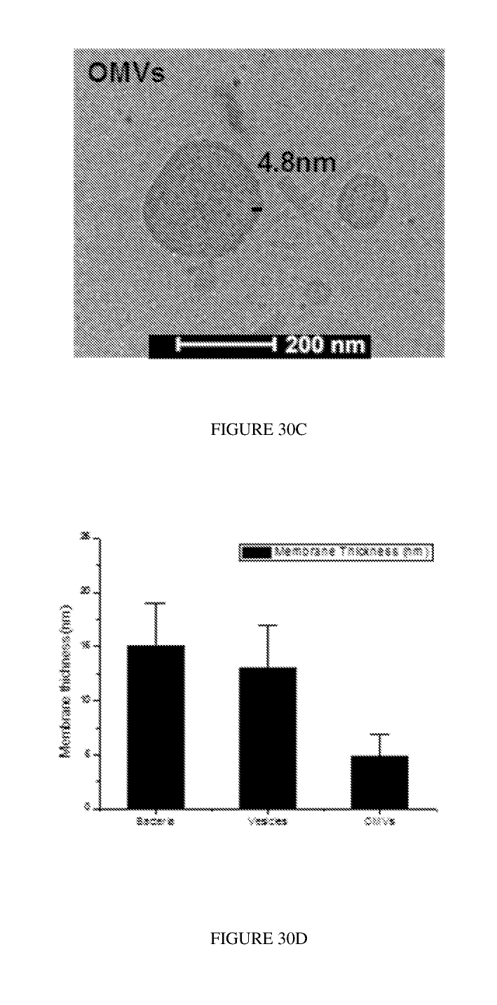

FIG. 29 shows the size of Pseudomonas aeruginosa cells compared to the vesicle compositions made from Pseudomonas aeruginosa cell membranes. As Pseudomonas aeruginosa is a gram-negative bacterium which comprises a double-layer membrane, cryo-TEM was used to visualize the unique structure of the formed vesicle compositions (see FIGS. 30A-30D). Unexpectedly, the vesicle compositions made from Pseudomonas aeruginosa cell membranes comprised double-layer membrane structures, which were akin to the Pseudomonas aeruginosa cells. This result indicates that the nitrogen cavitation formation approach described herein can preserve the intact membrane of the parent cells after the vesicle compositions are formed.

1. A vesicle composition comprising a cell membrane and an agent.

2. The vesicle composition of claim 1, wherein the vesicle composition further comprises a targeting molecule.

3. The vesicle composition of claim 2, wherein the targeting molecule is an intact targeting molecule.

4. The vesicle composition of claim 2, wherein the targeting molecule is a membrane molecule.

5.-91. (canceled)
